(19) United States
(12) Patent Application Publication
(10) Pub. No.: US 2003/0064945 A1
(43) Pub. Date: Apr. 3, 2003

(54) Enzymatic nucleic acid treatment of diseases or conditions related to levels of epidermal growth factor receptors

(76) Inventors: Saghir Akhtar, Safat (KW); James McSwiggen, Boulder, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/916,466

(22) Filed: Jul. 25, 2001

Related U.S. Application Data
(63) Continuation-in-part of application No. 09/848,754, filed on May 3, 2001.
Application No. 09/848,754 is a continuation-in-part of application No. 09/401,063, filed on Sep. 22, 1999.
Application No. 09/401,063 is a continuation of application No. 08/985,162, filed on Dec. 4, 1997, now Pat. No. 6,057,156.
(60) Provisional application No. 60/036,476, filed on Jan. 31, 1997.

(51) Int. Cl. A61K 48/00; C07H 21/02

(52) U.S. Cl. 514/44; 536/23.1

(57) ABSTRACT

The present invention relates to nucleic acid molecules, including antisense and enzymatic nucleic acid molecules, such as hammerhead ribozymes, DNAzymes, allozymes and antisense, which modulate the expression of epidermal growth factor receptor genes.

This patent application is a continuation-in-part of Akhtar et al., U.S. Ser. No. 09/848,754, filed May 3, 2001, entitled “ENZYMATIC NUCLEIC ACID TREATMENT OF DISEASES OR CONDITIONS RELATED TO LEVELS OF EPIDERMAL GROWTH FACTOR” which is a continuation application of Akhtar et al., U.S. Ser. No. 09/401,063, filed Sep. 22, 1999, entitled “ENZYMATIC NUCLEIC ACID TREATMENT OF DISEASES OR CONDITIONS RELATED TO LEVELS OF EPIDERMAL GROWTH FACTOR” which is a continuation application of Akhtar et al., U.S. Ser. No. 08/985,162, filed Dec. 4, 1997 now U.S. Pat. No. 6,057,156 entitled “ENZYMATIC NUCLEIC ACID TREATMENT OF DISEASES OR CONDITIONS RELATED TO LEVELS OF EPIDERMAL GROWTH FACTOR”, which claims priority from Akhtar et al., U.S. Ser. No. 60/036,749, filed Jan. 31, 1997, entitled “ENZYMATIC NUCLEIC ACID TREATMENT OF DISEASES OR CONDITIONS RELATED TO LEVELS OF EPIDERMAL GROWTH FACTOR”. These application are hereby incorporated by reference herein in their entirety including the drawings.

### BACKGROUND OF THE INVENTION

The present invention relates to therapeutic compositions and methods for the treatment or diagnosis of diseases or conditions related to EGFR expression levels, such as cancer. The discussion is not meant to be complete and is provided only for understanding of the invention that follows. This summary is not an admission that any of the work described below is prior art to the claimed invention.

The epidermal growth factor receptor (EGFR) is a 170 kDa transmembrane glycoprotein consisting of an extracellular ‘ligand’ binding domain, a transmembrane region and an intracellular domain with tyrosine kinase activity (Kung et al., 1994). The binding of growth factors to the EGFR results in down regulation of the ligand-receptor complex, autophosphorylation of the receptor and other protein substrates, leading ultimately to DNA synthesis and cell division. The external ligand binding domain is stimulated by EGF and also by TGFa, amphiregulin and some viral growth factors (Modjtahedi & Dean, 1994).

One of the striking characteristics of the EGFR gene (c-erbB1), located on chromosome 7, is it's homology to the avian erythroblastosis virus oncogene (v-erbB), which induces malignancies in chickens. The v-erbB gene codes for a truncated product that lacks the extracellular ligand binding domain. The tyrosine kinase domain of the EGFR has been found to have 97% homology to the v-erbB transforming protein (Downward et al., 1984).

Recent studies have shown that the EGFR is overexpressed in a number of malignant human tissues when compared to their normal tissue counterparts (for review see Khazaie et al., 1993). An important finding has been the discovery that the gene for the receptor is both amplified and overexpressed in a number of cancer cells. Overexpression of the EGFR is often accompanied by the co-expression of the growth factors EGF and TGF∝, suggesting that an autocrine pathway for control of growth may play a major part in the progression of tumors (Sporn & Roberts, 1985). It is now widely believed that this is a mechanism by which tumor cells can escape normal physiological control.

Growth factors and their receptors appear to have an important role in the development of human brain tumors. A high incidence of overexpression, amplification, deletion and structural rearrangement of the gene coding for the EGFR has been found in biopsies of brain tumors (Ostrowski et al., 1994). In fact the amplification of the EGFR gene in glioblastoma multiforme tumors is one of the most consistent genetic alterations known, with the EGFR being overexpressed in approximately 40% of malignant gliomas (Black, 1991). It has also been demonstrated that in 50% of glioblastomas, amplification of the EGFR gene is accompanied by the co-expression of mRNA for at least one or both of the growth factors EGF and TNFα(Ekstrand et al., 1991).

The amplified genes are frequently rearranged and associated with polymorphism leading to abnormal protein products (Wong et al., 1994). The rearrangements that have been characterized usually show deletions of part of the extracellular domain, resulting in the production of an EGFR protein that is smaller in size. Three classes of deletion mutant EGF receptor genes have been identified in glioblastoma tumors. Type I mutants lack the majority of the external domain, including the ligand binding site, type II mutants have a deletion in the domain adjacent to the membrane but can still bind ligands and type III, which is the most common and found in 17% of glioblastomas, have a deletion of 267 amino acids spanning domains I and II of the EGFR.

In addition to glioblastomas, abnormal EGFR expression has also been reported in a number of squamous epidermoid cancers and breast cancers (reviewed in Kung et al, 1994; Modjtahedi & Dean, 1994). Interestingly, evidence also suggests that many patients with tumors that overexpress the EGFR have a poorer prognosis than those who do not (Khazaie et al., 1993). Consequently, therapeutic strategies which can potentially inhibit or reduce the aberrant expression of the EGFR receptor are of great interest as potential anti-cancer agents.

Akhtar et al., U.S. Pat. No. 6,057,156, describe enzymatic nucleic acid molecules targeting epidermal growth factor receptors.

Akhtar et al., International PCT publication No. WO 98/33893, describe enzymatic nucleic acid molecules targeting epidermal growth factor receptors.

Halatsch et al., 2000, J. Neurosurg., 92, 297-305, describe specific hairpin ribozymes targeting specific epidermal growth factor receptors.

Yamazaki et al., 1998, J. Natl. Cancer Inst., 90, 581-587, describe specific hammerhead ribozymes targeting specific epidermal growth factor receptors.

Fell et al., 1997, Antisense Nucleic Acid Drug Dev., 7, 319-326, describe 2′-amino and 2′-O-methyl modified chimeric hammerhead ribozymes targeting epidermal growth factor receptor mRNA.

Yamazaki et al., 1995, PAACREAM, 36, 449, abstract No. 2556, describes a plasmid vector expressed hammerhead ribozyme targeted against a specific target site withing a specific mutant EGFR RNA.

Ludwig and Sproat, International PCT Publication No. WO 97/18312, describe a ribozyme with specific chemical modifications targeting EGFR.

Pyle and Michels, International PCT Publication No. WO 96/22689, describe specific group II intron based ribozymes targeting EGFR.

### SUMMARY OF THE INVENTION

The invention features novel nucleic acid-based molecules, for example, enzymatic nucleic acid molecules, allozymes, antisense nucleic acids, 2-5A antisense chimeras, triplex forming oligonucleotides, decoy RNA, dsRNA, siRNA, aptamers, and antisense nucleic acids containing RNA cleaving chemical groups, and methods to modulate gene expression, for example, genes encoding epidermal growth factor receptors. In particular, the instant invention features nucleic-acid based molecules and methods to modulate the expression of epidermal growth factor receptors (EGFR).

In one embodiment, the invention features one or more nucleic acid-based molecules and methods that independently or in combination modulate the expression of gene(s) encoding epidermal growth factor receptors. Specifically, the present invention features nucleic acid molecules that modulate the expression of EGFR genes HER1 (for example Genbank Accession No. NM_{—}005228), HER2 (for example Genbank Accession No. NM_{—}004448), HER3 (for example Genbank Accession No. NM_{—}001982), and HER4 (for example Genbank Accession No. NM_{—}005235).

The description below of the various aspects and embodiments is provided with reference to the exemplary epidermal growth receptor (EGFR) genes HER1, HER2, HER3, and HER4, collectively referred to hereinafter as EGFR. However, the various aspects and embodiments are also directed to other genes which express EGFR proteins and other receptors involved in oncogenesis. Those additional genes can be analyzed for target sites using the methods described for EGFR. Thus, the inhibition and the effects of such inhibition of the other genes can be performed as described herein.

In one embodiment, the invention features an enzymatic nucleic acid molecule which down regulates expression of an epidermal growth factor receptor (EGFR) gene, for example, wherein the EGFR gene comprises HER1, HER2, HER3, or HER4 and any combination thereof.

In another embodiment, the invention features an enzymatic nucleic acid molecule comprising a sequence selected from the group consisting of SEQ ID NOs: 215-432. In yet another embodiment, the invention features an enzymatic nucleic acid molecule comprising at least one binding arm wherein one or more of said binding arms comprises a sequence complementary to a sequence selected from the group consisting of SEQ ID NOs: 1-214.

In one embodiment, the invention features an antisense nucleic acid molecule comprising a sequence complementary to a sequence selected from the group consisting of SEQ ID NOs: 1-214.

In another embodiment, an enzymatic nucleic acid molecule, antisense nucleic acid molecule, 2-5A antisense chimera, triplex forming oligonucleotide, decoy RNA, dsRNA, siRNA, aptamer, or antisense nucleic acids containing RNA cleaving chemical groups of the invention is adapted to treat cancer.

In one embodiment, an enzymatic nucleic acid molecule of the invention has an endonuclease activity to cleave RNA encoded by an EGFR gene, for example, a HER1, HER2, HER3, or HER4 gene and any combination thereof.

In another embodiment, an enzymatic nucleic acid molecule of the invention is in a hammerhead, Inozyme, Zinzyme, DNAzyme, Amberzyme, or G-cleaver configuration.

In another embodiment, an enzymatic nucleic acid molecule of the invention having a hammerhead configuration comprises a sequence complementary to a sequence having SEQ ID NOs: 8, 24, 30, 36, 48, 53, 62, 70, 71, 81, 91, 118, 126, 133, 139, 152, 157, 160, 168, 175, 182, 192, 198, 201, and 210. In yet another embodiment, an enzymatic nucleic acid molecule of invention having a hammerhead configuration comprises a sequence having SEQ ID NOs: 215-239.

In another embodiment, an enzymatic nucleic acid molecule of the invention having an Inozyme configuration comprises a sequence complementary to a sequence having SEQ ID NOs: 1, 6, 14, 18, 20, 23, 25, 33, 35, 39, 42, 43, 47, 55, 68, 72, 93, 106, 112, 117, 119, 136, 137, 141, 148, 154, 155, 166, 188, 199, 200, and 206. In yet another embodiment, an enzymatic nucleic acid molecule of invention having an Inozyme configuration comprises a sequence having SEQ ID NOs: 240-271.

In another embodiment, an enzymatic nucleic acid molecule of the invention having a Zinzyme configuration comprises a sequence complementary to a sequence having SEQ ID NOs: 4, 22, 52, 54, 58, 63, 73, 76, 83, 85, 88, 101, 104, 108, 116, 131, 134, 142, 147, 149, 151, 158, 162, 167, 177, 183, 194, 197, 212, and 213. In yet another embodiment, an enzymatic nucleic acid molecule of invention having a Zinzyme configuration comprises a sequence having SEQ ID NOs: 272-301.

In another embodiment, an enzymatic nucleic acid molecule of the invention having a DNAzyme configuration comprises a sequence complementary to a sequence having SEQ ID NOs: 4, 5, 10, 11, 18, 21, 22, 34, 49, 51, 52, 54, 55, 58, 59, 63, 65, 71, 73, 75, 76, 77, 80, 83, 85, 88, 97, 101, 103, 104, 108, 116, 131, 133, 134, 136, 140, 142, 144, 145, 147, 149, 151, 158, 162, 164, 167, 173, 177, 183, 189, 190, 194, 197, 212, and 213. In yet another embodiment, an enzymatic nucleic acid molecule of invention having a DNAzyme configuration comprises a sequence having SEQ ID NOs: 302-357.

In another embodiment, an enzymatic nucleic acid molecule of the invention having an Amberzyme configuration comprises a sequence complementary to a sequence having SEQ ID NOs: 4, 9, 15, 17, 22, 26, 29, 41, 50, 52, 54, 56, 58, 60, 63, 66, 67, 73, 76, 83, 85, 86, 87, 88, 89, 94, 96, 98, 101, 104, 108, 109, 114, 115, 116, 120, 122, 123, 128, 129, 130, 131, 134, 135, 142, 143, 147, 149, 150, 151, 153, 156, 158, 159, 161, 162, 163, 167, 170, 176, 177, 178, 180, 1783, 184, 187, 194, 195, 197, 207, 208, 211-214. In yet another embodiment, an enzymatic nucleic acid molecule of invention having an Amberzyme configuration comprises a sequence having SEQ ID NOs: 358-432.

In one embodiment, an enzymatic nucleic acid molecule of the invention comprises between 8 and 100 bases complementary to the RNA of EGFR gene. In another embodiment, an enzymatic nucleic acid molecule of the invention comprises between 14 and 24 bases complementary to the RNA of EGFR gene.

In one embodiment, an enzymatic nucleic acid molecule, antisense nucleic acid molecule, 2-5A antisense chimera, triplex forming oligonucleotide, decoy RNA, dsRNA, siRNA, aptamer, or antisense nucleic acids containing RNA cleaving chemical groups of the invention is chemically synthesized.

In another embodiment, an enzymatic nucleic acid molecule, antisense nucleic acid molecule, 2-5A antisense chimera, triplex forming oligonucleotide, decoy RNA, dsRNA, siRNA, aptamer, or antisense nucleic acids containing RNA cleaving chemical groups of the invention comprises at least one 2′-sugar modification.

In another embodiment, an enzymatic nucleic acid molecule, antisense nucleic acid molecule, 2-5A antisense chimera, triplex forming oligonucleotide, decoy RNA, dsRNA, siRNA, aptamer, or antisense nucleic acids containing RNA cleaving chemical groups of the invention comprises at least one nucleic acid base modification.

In another embodiment, an enzymatic nucleic acid molecule, antisense nucleic acid molecule, 2-5A antisense chimera, triplex forming oligonucleotide, decoy RNA, dsRNA, siRNA, aptamer, or antisense nucleic acids containing RNA cleaving chemical groups of the invention comprises at least one phosphate backbone modification.

In one embodiment, the invention features a mammalian cell, for example a human cell, including the enzymatic nucleic acid molecule of the invention.

In another embodiment, the invention features a method of reducing EGFR expression or activity in a cell, comprising contacting the cell with an enzymatic nucleic acid molecule of the invention, under conditions suitable for the reduction.

In another embodiment, the invention features a method of reducing EGFR expression or activity in a cell, comprising the step of contacting the cell with an antisense nucleic acid molecule of the invention under conditions suitable for the reduction.

In yet another embodiment, the invention features a method of treatment of a patient having a condition associated with the level of EGFR, comprising contacting cells of the patient with an enzymatic nucleic acid molecule of the invention, under conditions suitable for the treatment.

In one embodiment, the invention features a method of treatment of a patient having a condition associated with the level of EGFR, comprising contacting cells of the patient with an antisense nucleic acid molecule of the invention, under conditions suitable for the treatment.

In another embodiment, a method of treatment of a patient having a condition associated with the level of EFGR is featured, wherein the method further comprises the use of one or more drug therapies under conditions suitable for the treatment.

For example, in one embodiment, the invention features a method for treatment of cancer, for example, breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, lymphoma, glioma, or multidrug resistant cancer under conditions suitable for the treatment.

In another embodiment, the invention features a method of cleaving RNA of EGFR gene comprising contacting an enzymatic nucleic acid molecule of the invention with the RNA of EGFR gene under conditions suitable for the cleavage, for example, wherein the cleavage is carried out in the presence of a divalent cation, such as Mg²⁺.

In one embodiment, an enzymatic nucleic acid molecule of the invention comprises a cap structure, for example a 3′,3′-linked or 5′,5′-linked deoxyabasic ribose derivative, wherein the cap structure is at the 5′-end, or 3′-end, or both the 5′-end and the 3′-end of the enzymatic nucleic acid molecule.

In another embodiment, an antisense nucleic acid molecule of the invention comprises a cap structure, for example a 3′,3′-linked or 5′,5′-linked deoxyabasic ribose derivative, wherein the cap structure is at the 5′-end, or 3′-end, or both the 5′-end and the 3′-end of the antisense nucleic acid molecule.

In one embodiment, the invention features an expression vector comprising a nucleic acid sequence encoding at least one enzymatic nucleic acid molecule of the invention, in a manner which allows expression of the nucleic acid molecule.

In another embodiment, the invention features a mammalian cell, for example, a human cell, including an expression vector of the invention.

In yet another embodiment, the expression vector of the invention further comprises a sequence for an antisense nucleic acid molecule complementary to the RNA of an EGFR gene.

In one embodiment, an expression vector of the invention comprises a nucleic acid sequence encoding two or more enzymatic nucleic acid molecules, which can be the same or different.

In another embodiment, the invention features a method for treatment of cancer, for example breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, lymphoma, glioma, or multidrug resistant cancer, comprising administering to a patient an enzymatic nucleic acid molecule, antisense nucleic acid molecule, 2-5A antisense chimera, triplex forming oligonucleotide, decoy RNA, dsRNA, siRNA, aptamer, or antisense nucleic acid containing RNA cleaving chemical groups of the invention, under conditions suitable for the treatment, including administering to the patient one or more other therapies, for example, monoclonal antibodies, EGFR-specific tyrosine kinase inhibitors, or chemotherapy.

In one embodiment, the method of treatment features an enzymatic nucleic acid molecule or antisense nucleic acid molecule of the invention comprises at least five ribose residues, at least ten 2′-O-methyl modifications, and a 3′-end modification, such as a 3′-3′ inverted abasic moiety. In another embodiment, an enzymatic nucleic acid molecule or antisense nucleic acid molecule of the invention further comprises phosphorothioate linkages on at least three of the 5′ terminal nucleotides.

In another embodiment, the method of treatment features monoclonal antibodies comprising mAB IMC C225 and/or mAB ABX-EGF. In yet another embodiment, the method features EGFR-specific tyrosine kinase inhibitors comprising OSI-774 and/or ZD1839.

In one embodiment, the method of treatment features chemotherapies comprising paclitaxel, docetaxel, cisplatin, methotrexate, cyclophosphamide, doxorubin, fluorouracil carboplatin, edatrexate, gemcitabine, or vinorelbine, as well as combinations thereof.

In another embodiment, the invention features a method of administering to a mammal, for example a human, an enzymatic nucleic acid molecule, antisense nucleic acid molecule, 2-5A antisense chimera, triplex forming oligonucleotide, decoy RNA, dsRNA, siRNA, aptamer, or antisense nucleic acid containing RNA cleaving chemical groups of the invention, comprising contacting the mammal with the nucleic acid molecule under conditions suitable for the administration, for example, in the presence of a delivery reagent such as a lipid, cationic lipid, phospholipid, or liposome.

In yet another embodiment, the invention features a method of administering to a mammal an enzymatic nucleic acid molecule, antisense nucleic acid molecule, 2-5A antisense chimera, triplex forming oligonucleotide, decoy RNA, dsRNA, siRNA, aptamer, or antisense nucleic acid containing RNA cleaving chemical groups of the invention in conjunction with a chemotherapeutic agent, comprising contacting the mammal, for example a human, with the nucleic acid molecule and the chemotherapeutic agent under conditions suitable for the administration.

In one embodiment, the invention features the use of an enzymatic nucleic acid molecule, preferably in the hammerhead, NCH, G-cleaver, Amberzyme, Zinzyme, and/or DNAzyme motif, to down-regulate the expression of EGFR genes.

By “inhibit”, “down-regulate”, or “reduce”, it is meant that the expression of the gene, or level of RNAs or equivalent RNAs encoding one or more protein subunits, or activity of one or more protein subunits, such as EGFR subunit(s), is reduced below that observed in the absence of the nucleic acid molecules of the invention. In one embodiment, inhibition. down-regulation or reduction with enzymatic nucleic acid molecule preferably is below that level observed in the presence of an enzymatically inactive or attenuated molecule that is able to bind to the same site on the target RNA, but is unable to cleave that RNA. In another embodiment, inhibition, down-regulation, or reduction with antisense oligonucleotides is preferably below that level observed in the presence of, for example, an oligonucleotide with scrambled sequence or with mismatches. In another embodiment, inhibition, down-regulation, or reduction of EGFR with the nucleic acid molecule of the instant invention is greater in the presence of the nucleic acid molecule than in its absence.

By “up-regulate” is meant that the expression of the gene, or level of RNAs or equivalent RNAs encoding one or more protein subunits, or activity of one or more protein subunits, such as EGFR subunit(s), is greater than that observed in the absence of the nucleic acid molecules of the invention. For example, the expression of a gene, such as EGFR gene, can be increased in order to treat, prevent, ameliorate, or modulate a pathological condition caused or exacerbated by an absence or low level of gene expression.

By “modulate” is meant that the expression of the gene, or level of RNAs or equivalent RNAs encoding one or more protein subunits, or activity of one or more protein subunit(s) is up-regulated or down-regulated, such that the expression, level, or activity is greater than or less than that observed in the absence of the nucleic acid molecules of the invention.

By “enzymatic nucleic acid molecule” it is meant a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% can also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids can be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving and/or ligation activity to the molecule (Cech et al., U.S. Pat. No. 4,987,071; Cech et al., 1988, 260 JAMA 3030).

By “nucleic acid molecule” as used herein is meant a molecule having nucleotides. The nucleic acid can be single, double, or multiple stranded and can comprise modified or unmodified nucleotides or non-nucleotides or various mixtures and combinations thereof.

By “enzymatic portion” or “catalytic domain” is meant that portion/region of the enzymatic nucleic acid molecule essential for cleavage of a nucleic acid substrate (for example see FIG. 1).

By “substrate binding arm” or “substrate binding domain” is meant that portion/region of a enzymatic nucleic acid which is able to interact, for example via complementarity (i.e., able to base-pair with), with a portion of its substrate. Preferably, such complementarity is 100%, but can be less if desired. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). Examples of such arms are shown generally in FIGS. 1-4. That is, these arms contain sequences within a enzymatic nucleic acid which are intended to bring enzymatic nucleic acid and target RNA together through complementary base-pairing interactions. The enzymatic nucleic acid of the invention can have binding arms that are contiguous or non-contiguous and may be of varying lengths. The length of the binding arm(s) are preferably greater than or equal to four nucleotides and of sufficient length to stably interact with the target RNA; preferably 12-100 nucleotides; more preferably 14-24 nucleotides long (see for example Werner and Uhlenbeck, supra; Hamman et al., supra; Hampel et al., EP0360257; Berzal-Herrance et al., 1993, EMBO J., 12, 2567-73) or between 8 and 14 nucleotides long. If two binding arms are chosen, the design is such that the length of the binding arms are symmetrical (i.e., each of the binding arms is of the same length; e.g., four and four, five and five nucleotides, or six and six nucleotides, or seven and seven nucleotides long) or asymmetrical (i.e., the binding arms are of different length; e.g., three and five, six and three nucleotides; three and six nucleotides long; four and five nucleotides long; four and six nucleotides long; four and seven nucleotides long; and the like).

By “Inozyme” or “NCH” motif or configuration is meant, an enzymatic nucleic acid molecule comprising a motif as is generally described as NCH Rz in FIG. 2. Inozymes possess endonuclease activity to cleave RNA substrates having a cleavage triplet NCH/, where N is a nucleotide, C is cytidine and H is adenosine, uridine or cytidine, and/represents the cleavage site. H is used interchangeably with X. Inozymes can also possess endonuclease activity to cleave RNA substrates having a cleavage triplet NCN/, where N is a nucleotide, C is cytidine, and/represents the cleavage site. “I” in FIG. 2 represents an Inosine nucleotide, preferably a ribo-Inosine or xylo-Inosine nucleoside.

By “G-cleaver” motif or configuration is meant, an enzymatic nucleic acid molecule comprising a motif as is generally described as G-cleaver Rz in FIG. 2. G-cleavers possess endonuclease activity to cleave RNA substrates having a cleavage triplet NYN/, where N is a nucleotide, Y is uridine or cytidine and/represents the cleavage site. G-cleavers can be chemically modified as is generally shown in FIG. 2.

By “amberzyme” motif or configuration is meant, an enzymatic nucleic acid molecule comprising a motif as is generally described in FIG. 3. Amberzymes possess endonuclease activity to cleave RNA substrates having a cleavage triplet NG/N, where N is a nucleotide, G is guanosine, and/represents the cleavage site. Amberzymes can be chemically modified to increase nuclease stability through substitutions as are generally shown in FIG. 3. In addition, differing nucleoside and/or non-nucleoside linkers can be used to substitute the 5′-gaaa-3′ loops shown in the figure. Amberzymes represent a non-limiting example of an enzymatic nucleic acid molecule that does not require a ribonucleotide (2′-OH) group within its own nucleic acid sequence for activity.

By “zinzyme” motif or configuration is meant, an enzymatic nucleic acid molecule comprising a motif as is generally described in FIG. 4. Zinzymes possess endonuclease activity to cleave RNA substrates having a cleavage triplet including but not limited to YG/Y, where Y is uridine or cytidine, and G is guanosine and/represents the cleavage site. Zinzymes can be chemically modified to increase nuclease stability through substitutions as are generally shown in FIG. 4, including substituting 2′-O-methyl guanosine nucleotides for guanosine nucleotides. In addition, differing nucleotide and/or non-nucleotide linkers can be used to substitute the 5′-gaaa-2′ loop shown in the figure. Zinzymes represent a non-limiting example of an enzymatic nucleic acid molecule that does not require a ribonucleotide (2′-OH) group within its own nucleic acid sequence for activity.

By ‘DNAzyme’ is meant, an enzymatic nucleic acid molecule that does not require the presence of a 2′-OH group within its own nucleic acid sequence for activity. In particular embodiments the enzymatic nucleic acid molecule can have an attached linker or linkers or other attached or associated groups, moieties, or chains containing one or more nucleotides with 2′-OH groups. DNAzymes can be synthesized chemically or expressed endogenously in vivo, by means of a single stranded DNA vector or equivalent thereof. An example of a DNAzyme is shown in FIG. 5 and is generally reviewed in Usman et al., U.S. Pat. No., 6,159,714; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. The “10-23” DNAzyme motif is one particular type of DNAzyme that was evolved using in vitro selection (see Santoro et al., supra). Additional DNAzyme motifs can be selected for using techniques similar to those described in these references, and hence, are within the scope of the present invention.

By “sufficient length” is meant an oligonucleotide of greater than or equal to 3 nucleotides that is of a length great enough to provide the intended function under the expected condition. For example, for binding arms of enzymatic nucleic acid “sufficient length” means that the binding arm sequence is long enough to provide stable binding to a target site under the expected binding conditions. Preferably, the binding arms are not so long as to prevent useful turnover of the nucleic acid molecule.

By “stably interact” is meant interaction of the oligonucleotides with target nucleic acid (e.g., by forming hydrogen bonds with complementary nucleotides in the target under physiological conditions) that is sufficient to the intended purpose (e.g., cleavage of target RNA by an enzyme).

By “equivalent” RNA to EGFR is meant to include RNA molecules having homology (partial or complete) to RNA encoding EGFR proteins or encoding proteins with similar function as EGFR proteins in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like.

By “homology” is meant the nucleotide sequence of two or more nucleic acid molecules is partially or completely identical.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

By “RNase H activating region” is meant a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothioate (preferably at least four of the nucleotides are phosphorothiote substitutions; more specifically, 4-11 of the nucleotides are phosphorothiote substitutions); phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

By “2-5A antisense chimera” is meant an antisense oligonucleotide containing a 5′-phosphorylated 2′-5′-linked adenylate residue. These chimeras bind to target RNA in a sequence-specific manner and activate a cellular 2-5A-dependent ribonuclease which, in turn, cleaves the target RNA (Torrence et al., 1993 Proc. Natl. Acad. Sci. USA 90, 1300; Silverman et al., 2000, Methods Enzymol., 313, 522-533; Player and Torrence, 1998, Pharmacol. Ther., 78, 55-113).

By “triplex forming oligonucleotides” is meant an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504; Fox, 2000, Curr. Med. Chem., 7, 17-37; Praseuth et. al., 2000, Biochim. Biophys. Acta, 1489, 181-206).

By “gene” it is meant a nucleic acid that encodes an RNA, for example, nucleic acid sequences including but not limited to structural genes encoding a polypeptide.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” or “2′-OH” is meant a nucleotide with a hydroxyl group at the 2′ position of a β-D-ribo-furanose moiety.

By “decoy RNA” is meant an RNA molecule or aptamer that is designed to preferentially bind to a predetermined ligand. Such binding can result in the inhibition or activation of a target molecule. The decoy RNA or aptamer can compete with a naturally occurring binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is but a specific example and those in the art will recognize that other embodiments can be readily generated using techniques generally known in the art, see for example Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628. Similarly, a decoy RNA can be designed to bind to a EGFR receptor and block the binding of EGFR or a decoy RNA can be designed to bind to EGFR and prevent interaction with the EGFR receptor.

The term “double stranded RNA” or “dsRNA” as used herein refers to a double stranded RNA molecule capable of RNA interference, including short interfering RNA “siRNA” see for example Bass, 2001, Nature, 411, 428-429; Elbashir et al., 2001, Nature, 411, 494-498).

The term “allozyme” as used herein refers to an allosteric enzymatic nucleic acid molecule, see for example see for example George et al., U.S. Pat. Nos. 5,834,186 and 5,741,679, Shih et al., U.S. Pat. No. 5,589,332, Nathan et al., U.S. Pat. No 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842.The term “2-5A chimera” as used herein refers to an oligonucleotide containing a 5′-phosphorylated 2′-5′-linked adenylate residue. These chimeras bind to target RNA in a sequence-specific manner and activate a cellular 2-5A-dependent ribonuclease which, in turn, cleaves the target RNA (Torrence et al., 1993 Proc. Natl. Acad. Sci. USA 90, 1300; Silverman et al., 2000, Methods Enzymol., 313, 522-533; Player and Torrence, 1998, Pharmacol. Ther., 78, 55-113).

The term “triplex forming oligonucleotides” as used herein refers to an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504; Fox, 2000, Curr. Med. Chem., 7, 17-37; Praseuth et. al., 2000, Biochim. Biophys. Acta, 1489, 181-206).

Several varieties of naturally-occurring enzymatic RNAs are known presently. Each can catalyze the hydrolysis of RNA phosphodiester bonds in trans (and thus can cleave other RNA molecules) under physiological conditions. Table I summarizes some of the characteristics of these ribozymes. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

The enzymatic nucleic acid molecule that cleave the specified sites in EGFR-specific RNAs represent a novel therapeutic approach to treat a variety of cancers, including but not limited to breast, lung, prostate, colorectal, brain, esophageal, bladder, pancreatic, cervical, head and neck, and ovarian cancer, melanoma, lymphoma, glioma, multidrug resistant cancers, and/or other cancers which respond to the modulation of EGFR expression.

In one embodiment of the inventions described herein, the enzymatic nucleic acid molecule is formed in a hammerhead or hairpin motif, but can also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183; of hairpin motifs by Hampel et al., EP0360257, Hampel and Tritz, 1989 Biochemistry 28, 4929, Feldstein et al., 1989, Gene 82, 53, Haseloff and Gerlach, 1989, Gene, 82, 43, and Hampel et al., 1990 Nucleic Acids Res. 18, 299; Chowrira & McSwiggen, U.S. Pat. No. 5,631,359; of the hepatitis delta virus motif is described by Perrotta and Been, 1992 Biochemistry 31, 16; of the RNase P motif by Guerrier-Takada et al., 1983 Cell 35, 849; Forster and Altman, 1990, Science 249, 783; Li and Altman, 1996, Nucleic Acids Res. 24, 835; Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J. 14, 363); Group II introns are described by Griffin et al., 1995, Chem. Biol. 2, 761; Michels and Pyle, 1995, Biochemistry 34, 2965; Pyle et al., International PCT Publication No. WO 96/22689; of the Group I intron by Cech et al., U.S. Pat. No. 4,987,071 and of DNAzymes by Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262, and Beigelman et al., International PCT publication No. WO 99/55857. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs such as the Aptazyme (Breaker et al., WO 98/43993), Amberzyme (Class I motif; FIG. 3; Beigelman et al., U.S. Ser. No. 09/301,511) and Zinzyme (FIG. 4) (Beigelman et al., U.S. Ser. No. 09/301,511), all included by reference herein including drawings, can also be used in the present invention. These specific motifs or configurations are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

In one embodiment of the present invention, a nucleic acid molecule of the instant invention can be between 12 and 100 nucleotides in length. Exemplary enzymatic nucleic acid molecules of the invention are shown in Table III-VII. For example, enzymatic nucleic acid molecules of the invention are preferably between 15 and 50 nucleotides in length, more preferably between 25 and 40 nucleotides in length, e.g., 34, 36, or 38 nucleotides in length (for example see Jarvis et al., 1996, J. Biol. Chem., 271, 29107-29112). Exemplary DNAzymes of the invention are preferably between 15 and 40 nucleotides in length, more preferably between 25 and 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Exemplary antisense molecules of the invention are preferably between 15 and 75 nucleotides in length, more preferably between 20 and 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). Exemplary triplex forming oligonucleotide molecules of the invention are preferably between 10 and 40 nucleotides in length, more preferably between 12 and 25 nucleotides in length, e.g., 18, 19, 20, or 21 nucleotides in length (see for example Maher et al., 1990, Biochemistry, 29, 8820-8826; Strobel and Dervan, 1990, Science, 249, 73-75). Those skilled in the art will recognize that all that is required is for the nucleic acid molecule are of length and conformation sufficient and suitable for the nucleic acid molecule to catalyze a reaction contemplated herein. The length of the nucleic acid molecules of the instant invention are not limiting within the general limits stated.

In a preferred embodiment, a nucleic acid molecule that modulates, for example, down-regulates, EGFR replication or expression comprises between 8 and 100 bases complementary to a RNA molecule of EGFR. More preferably, a nucleic acid molecule that modulates EGFR replication or expression comprises between 14 and 24 bases complementary to a RNA molecule of EGFR.

The invention provides a method for producing a class of nucleic acid-based gene modulating agents which exhibit a high degree of specificity for the RNA of a desired target. For example, the enzymatic nucleic acid molecule is preferably targeted to a highly conserved sequence region of target RNAs encoding EGFR (specifically EGFR genes) such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., ribozymes and antisense) can be expressed from DNA and/or RNA vectors that are delivered to specific cells.

As used in herein “cell” is used in its usual biological sense, and does not refer to an entire multicellular organism. The cell can, for example, be in vitro, e.g., in cell culture, or present in a multicellular organism, including,, e.g., birds, plants and mammals such as humans, cows, sheep, apes, monkeys, swine, dogs, and cats. The cell may be prokaryotic (e.g., bacterial cell) or eukaryotic (e.g., mammalian or plant cell).

By “EGFR proteins” is meant, protein receptor or a mutant protein derivative thereof, having epidermal growth factor receptor activity, for example, having the ability to bind epidermal growth factor and/or having tyrosine kinase activity.

By “highly conserved sequence region” is meant, a nucleotide sequence of one or more regions in a target gene does not vary significantly from one generation to the other or from one biological system to the other.

Nucleic acid-based inhibitors of EGFR expression are useful for the prevention and/or treatment of cancers and cancerous conditions such as breast, lung, prostate, colorectal, brain, esophageal, bladder, pancreatic, cervical, head and neck, and ovarian cancer, melanoma, lymphoma, glioma, multidrug resistant cancers, and any other diseases or conditions that are related to or will respond to the levels of EGFR in a cell or tissue, alone or in combination with other therapies. The reduction of EGFR expression (specifically EGFR gene RNA levels) and thus reduction in the level of the respective protein relieves, to some extent, the symptoms of the disease or condition.

The nucleic acid-based inhibitors of the invention can be added directly, or can be complexed with cationic lipids, packaged within liposomes, or otherwise delivered to target cells or tissues. The nucleic acid or nucleic acid complexes can be locally administered to relevant tissues ex vivo, or in vivo through injection or infusion pump, with or without their incorporation in biopolymers. In preferred embodiments, the enzymatic nucleic acid inhibitors comprise sequences, which are complementary to the substrate sequences in Tables III to VII. Examples of such enzymatic nucleic acid molecules also are shown in Tables III to VII. Examples of such enzymatic nucleic acid molecules consist essentially of sequences defined in these tables.

In another embodiment, the invention features antisense nucleic acid molecules and 2-5A chimera including sequences complementary to the substrate sequences shown in Tables III to VII. Such nucleic acid molecules can include sequences as shown for the binding arms of the enzymatic nucleic acid molecules in Tables III to VII. Similarly, triplex molecules can be provided targeted to the corresponding DNA target regions, and containing the DNA equivalent of a target sequence or a sequence complementary to the specified target (substrate) sequence. Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both.

By “consists essentially of” is meant that the active nucleic acid molecule of the invention, for example, an enzymatic nucleic acid molecule, contains an enzymatic center or core equivalent to those in the examples, and binding arms able to bind RNA such that cleavage at the target site occurs. Other sequences can be present which do not interfere with such cleavage. Thus, a core region can, for example, include one or more loop, stem-loop structure, or linker which does not prevent enzymatic activity. Thus, the underlined regions in the sequences in Tables III and IV can be such a loop, stem-loop, nucleotide linker, and/or non-nucleotide linker and can be represented generally as sequence “X”. For example, a core sequence for a hammerhead enzymatic nucleic acid can comprise a conserved sequence, such as 5′-CUGAUGAG-3′ and 5′-CGAA-3′ connected by “X”, where X is 5′-GCCGUUAGGC-3′ (SEQ ID NO 446), or any other Stem II region known in the art, or a nucleotide and/or non-nucleotide linker. Similarly, for other nucleic acid molecules of the instant invention, such as Inozyme, G-cleaver, amberzyme, zinzyme, DNAzyme, antisense, 2-5A antisense, triplex forming nucleic acid, and decoy nucleic acids, other sequences or non-nucleotide linkers can be present that do not interfere with the function of the nucleic acid molecule.

Sequence X can be a linker of ≧2 nucleotides in length, preferably 3, 4, 5, 6, 7, 8, 9, 10, 15, 20, 26, 30, where the nucleotides can preferably be internally base-paired to form a stem of preferably ≧2 base pairs. Alternatively or in addition, sequence X can be a non-nucleotide linker. In yet another embodiment, the nucleotide linker X can be a nucleic acid aptamer, such as an ATP aptamer, HIV Rev aptamer (RRE), HIV Tat aptamer (TAR) and others (for a review see Gold et al., 1995, Annu. Rev. Biochem., 64, 763; and Szostak & Ellington, 1993, in The RNA World, ed. Gesteland and Atkins, pp. 511, CSH Laboratory Press). A “nucleic acid aptamer” as used herein is meant to indicate a nucleic acid sequence capable of interacting with a ligand. The ligand can be any natural or a synthetic molecule, including but not limited to a resin, metabolites, nucleosides, nucleotides, drugs, toxins, transition state analogs, peptides, lipids, proteins, amino acids, nucleic acid molecules, hormones, carbohydrates, receptors, cells, viruses, bacteria and others.

In yet another embodiment, the non-nucleotide linker X is as defined herein. The term “non-nucleotide” as used herein include either abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. A “non-nucleotide” further means any group or compound which can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine. Thus, in a preferred embodiment, the invention features an enzymatic nucleic acid molecule having one or more non-nucleotide moieties, and having enzymatic activity to cleave an RNA or DNA molecule.

In another aspect of the invention, enzymatic nucleic acid molecules or antisense molecules that interact with target RNA molecules and down-regulate EGFR (specifically EGFR gene) activity are expressed from transcription units inserted into DNA or RNA vectors. The recombinant vectors are preferably DNA plasmids or viral vectors. Enzymatic nucleic acid molecule or antisense expressing viral vectors can be constructed based on, but not limited to, adeno-associated virus, retrovirus, adenovirus, or alphavirus. Preferably, the recombinant vectors capable of expressing the enzymatic nucleic acid molecules or antisense are delivered as described above, and persist in target cells. Alternatively, viral vectors can be used that provide for transient expression of enzymatic nucleic acid molecules or antisense. Such vectors can be repeatedly administered as necessary. Once expressed, the enzymatic nucleic acid molecules or antisense bind to the target RNA and down-regulate its function or expression. Delivery of enzymatic nucleic acid molecule or antisense expressing vectors can be systemic, such as by intravenous or intramuscular administration, by administration to target cells ex-planted from the patient followed by reintroduction into the patient, or by any other means that would allow for introduction into the desired target cell. Antisense DNA can be expressed via the use of a single stranded DNA intracellular expression vector.

By “vectors” is meant any nucleic acid- and/or viral-based technique used to deliver a desired nucleic acid.

By “patient” is meant an organism, which is a donor or recipient of explanted cells, or the cells themselves. “Patient” also refers to an organism to which the nucleic acid molecules of the invention can be administered. Preferably, a patient is a mammal or mammalian cells. More preferably, a patient is a human or human cells.

By “enhanced enzymatic activity” is meant to include activity measured in cells and/or in vivo where the activity is a reflection of both the catalytic activity and the stability of the nucleic acid molecules of the invention. In this invention, the product of these properties can be increased in vivo compared to an all RNA enzymatic nucleic acid or all DNA enzyme. In some cases, the activity or stability of the nucleic acid molecule can be decreased (i.e., less than ten-fold), but the overall activity of the nucleic acid molecule is enhanced, in vivo.

The nucleic acid molecules of the instant invention, individually, or in combination or in conjunction with other drugs, can be used to treat diseases or conditions discussed above. For example, to treat a disease or condition associated with the levels of EGFR, the patient can be treated, or other appropriate cells can be treated, as is evident to those skilled in the art, individually or in combination with one or more drugs under conditions suitable for the treatment.

In a further embodiment, the described molecules, such as antisense or ribozymes, can be used in combination with other known treatments to treat conditions or diseases discussed above. For example, the described molecules can be used in combination with one or more known therapeutic agents to treat breast, lung, prostate, colorectal, brain, esophageal, bladder, pancreatic, cervical, head and neck, and ovarian cancer, melanoma, lymphoma, glioma, multidrug resistant cancers, and/or other cancers which respond to the modulation of EGFR expression.

In another embodiment, the invention features nucleic acid-based inhibitors (e.g., enzymatic nucleic acid molecules (eg; ribozymes), antisense nucleic acids, 2-5A antisense chimeras, triplex DNA, antisense nucleic acids containing RNA cleaving chemical groups) and methods for their use to down regulate or inhibit the expression of genes (e.g., EGFR) capable of progression and/or maintenance of cancer, and/or other disease states which respond to the modulation of EGFR expression.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present.

Other features and advantages of the invention will be apparent from the following description of the preferred embodiments thereof, and from the claims.

### BRIEF DESCRIPTION OF THE DRAWINGS

FIG. 1 shows the secondary structure model for seven different classes of enzymatic nucleic acid molecules. Arrow indicates the site of cleavage. --------- indicate the target sequence. Lines interspersed with dots are meant to indicate tertiary interactions. - is meant to indicate base-paired interaction. Group I Intron: P1-P9.0 represent various stem-loop structures (Cech et al., 1994, Nature Struc. Bio., 1, 273). RNase P (M1RNA): EGS represents external guide sequence (Forster et al., 1990, Science, 249, 783; Pace et al., 1990, J. Biol. Chem., 265, 3587). Group II Intron: 5′SS means 5′ splice site; 3′SS means 3′-splice site; IBS means intron binding site; EBS means exon binding site (Pyle et al., 1994, Biochemistry, 33, 2716). VS RNA: I-VI are meant to indicate six stem-loop structures; shaded regions are meant to indicate tertiary interaction (Collins, International PCT Publication No. WO 96/19577). HDV Ribozyme: : I-IV are meant to indicate four stem-loop structures (Been et al., U.S. Pat. No. 5,625,047). Hammerhead Ribozyme: I-III are meant to indicate three stem-loop structures; stems I-III can be of any length and can be symmetrical or asymmetrical (Usman et al., 1996, Curr. Op. Struct. Bio., 1, 527). Hairpin Ribozyme: Helix 1, 4 and 5 can be of any length; Helix 2 is between 3 and 8 base-pairs long; Y is a pyrimidine; Helix 2 (H2) is provided with a least 4 base pairs (i.e., n is 1, 2, 3 or 4) and helix 5 can be optionally provided of length 2 or more bases (preferably 3-20 bases, i.e., m is from 1-20 or more). Helix 2 and helix 5 can be covalently linked by one or more bases (i.e., r is ≧1 base). Helix 1, 4 or 5 can also be extended by 2 or more base pairs (e.g., 4-20 base pairs) to stabilize the ribozyme structure, and preferably is a protein binding site. In each instance, each N and N′ independently is any normal or modified base and each dash represents a potential base-pairing interaction. These nucleotides can be modified at the sugar, base or phosphate. Complete base-pairing is not required in the helices, but is preferred. Helix 1 and 4 can be of any size (i.e., o and p is each independently from 0 to any number, e.g., 20) as long as some base-pairing is maintained. Essential bases are shown as specific bases in the structure, but those in the art will recognize that one or more can be modified chemically (abasic, base, sugar and/or phosphate modifications) or replaced with another base without significant effect. Helix 4 can be formed from two separate molecules, i.e., without a connecting loop. The connecting loop when present can be a ribonucleotide with or without modifications to its base, sugar or phosphate. “q” ≧ is 2 bases. The connecting loop can also be replaced with a non-nucleotide linker molecule. H refers to bases A, U, or C. Y refers to pyrimidine bases. (Burke et al., 1996, Nucleic Acids & Mol. Biol., 10, 129; Chowrira et al., U.S. Pat. No. 5,631,359).

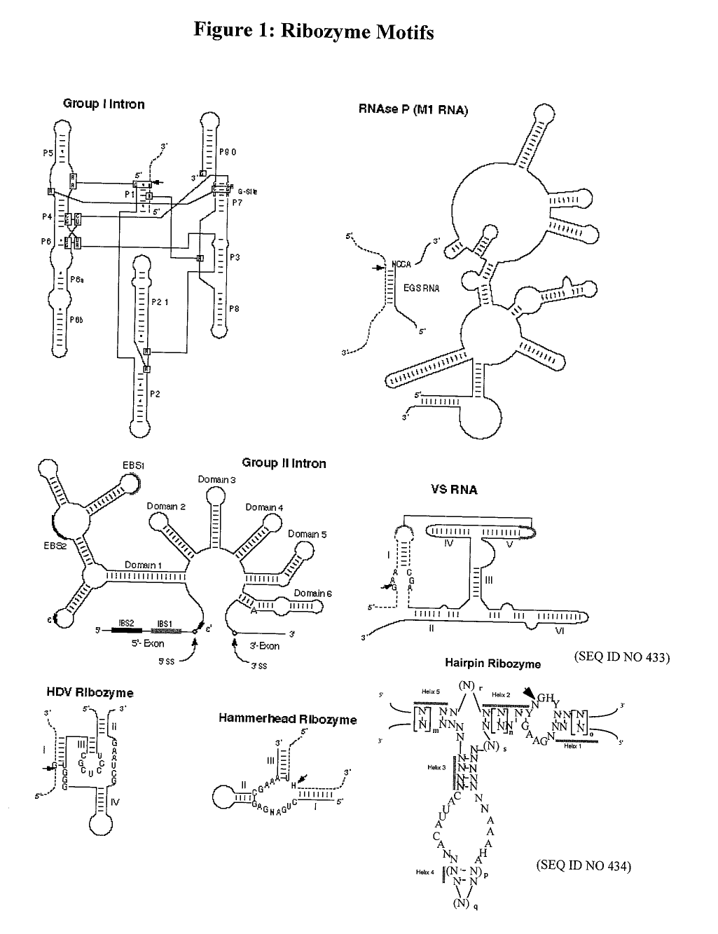

FIG. 2 shows examples of chemically stabilized ribozyme motifs. HH Rz, represents hammerhead ribozyme motif (Usman et al., 1996, Curr. Op. Struct. Bio., 1, 527); NCH Rz represents the NCH ribozyme motif (Ludwig & Sproat, International PCT Publication No. WO 98/58058); G-Cleaver, represents G-cleaver ribozyme motif (Kore et al., 1998, Nucleic Acids Research 26, 4116-4120, Eckstein et al., International PCT publication No. WO 99/16871). N or n, represent independently a nucleotide which can be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

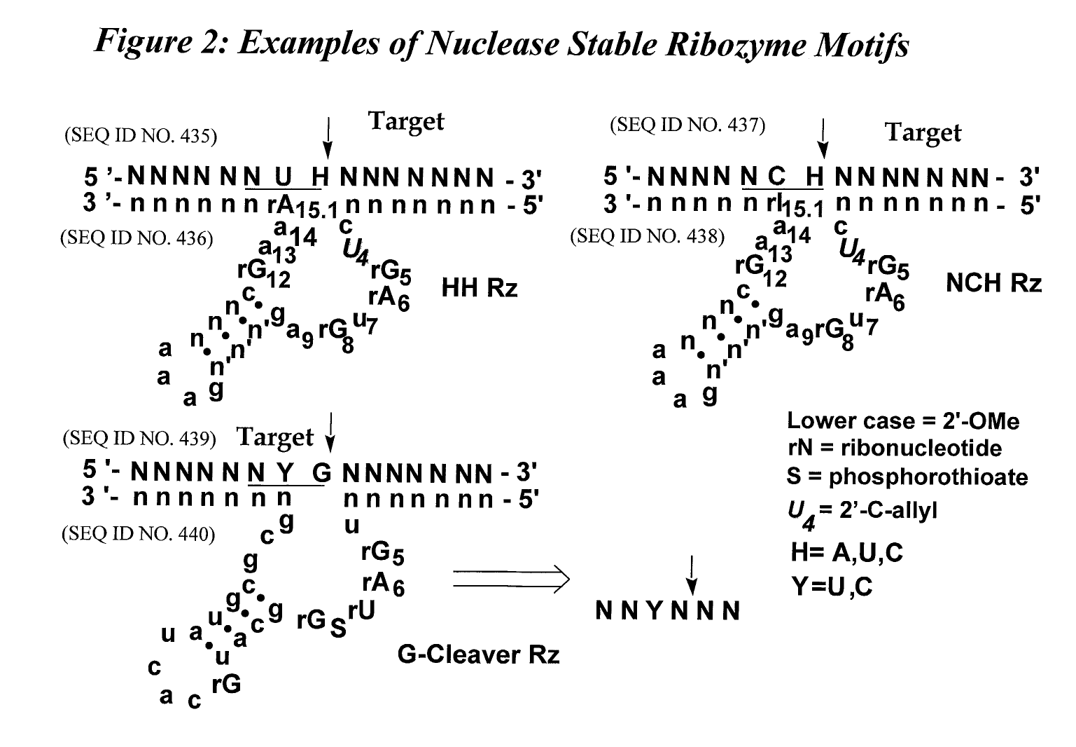

FIG. 3 shows an example of the Amberzyme ribozyme motif that is chemically stabilized (see for example Beigelman et al., International PCT publication No. WO 99/55857).

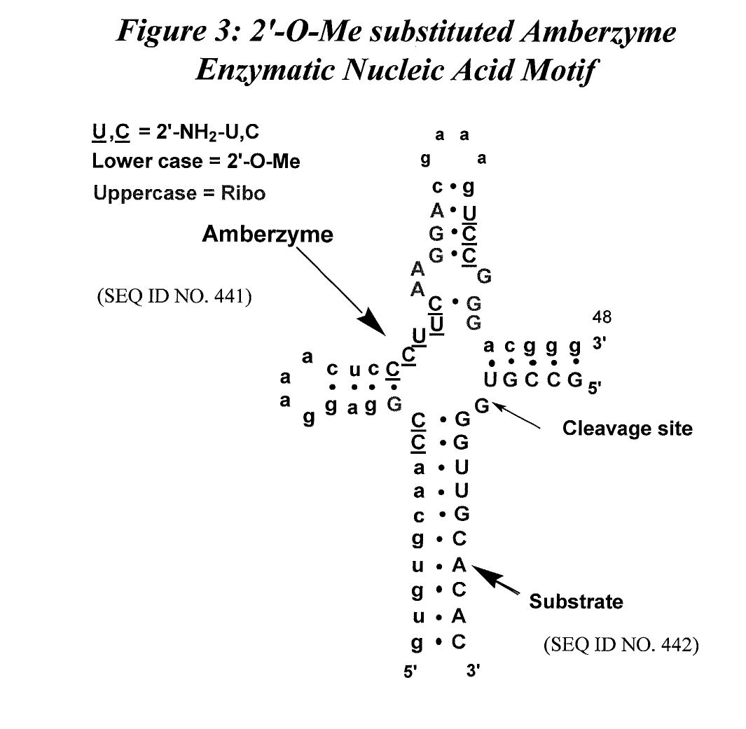

FIG. 4 shows an example of the Zinzyme A ribozyme motif that is chemically stabilized (see for example Beigelman et al., Beigelman et al., International PCT publication No. WO 99/55857).

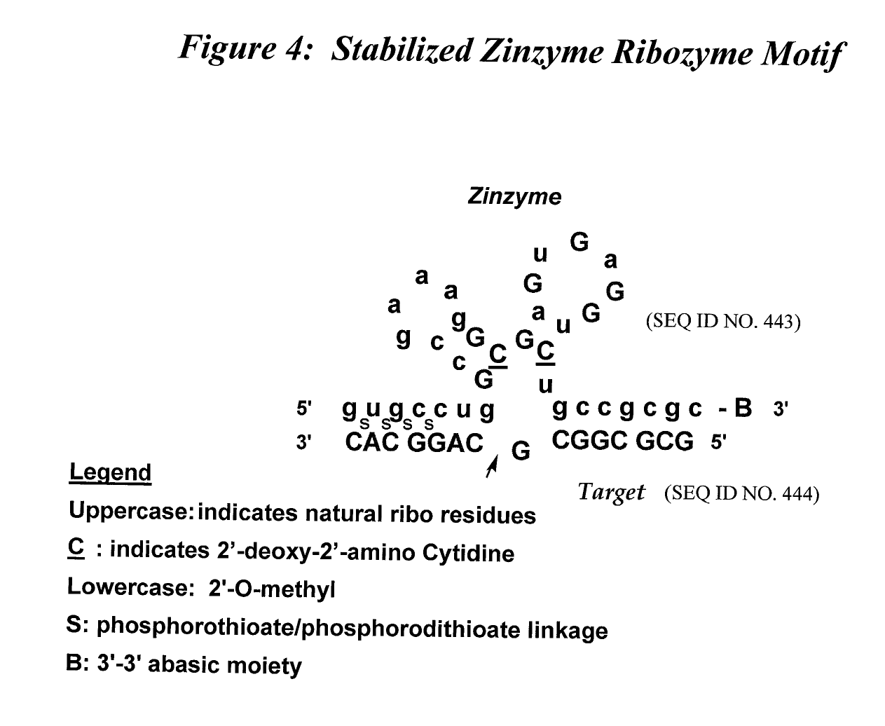

FIG. 5 shows an example of a DNAzyme motif described by Santoro et al., 1997, PNAS, 94, 4262.

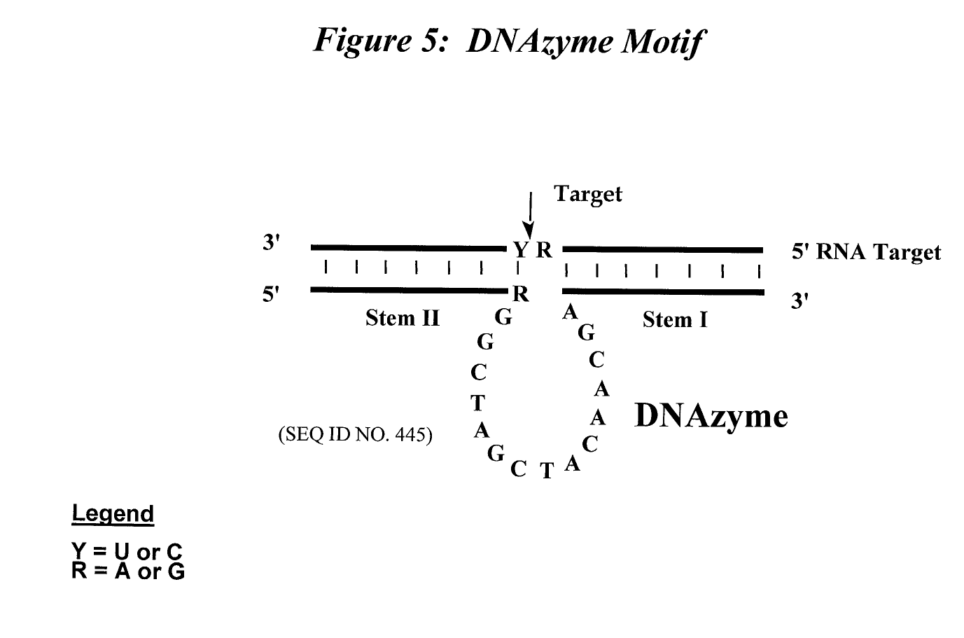

### DESCRIPTION OF THE PREFERRED EMBODIMENTS

Nucleic Acid Molecules and Mechanism of Action

Antisense: Antisense molecules can be modified or unmodified RNA, DNA, or mixed polymer oligonucleotides and primarily function by specifically binding to matching sequences resulting in inhibition of peptide synthesis (Wu-Pong, November 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA can result in nuclease degradation of the heteroduplex (Wu-Pong, supra; Crooke, supra). To date, the only backbone modified DNA chemistry which act as substrates for RNase H are phosphorothioates, phosphorodithioates, and borontrifluoridates. Recently it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., U.S. Ser. No. 60/101,174 which was filed on Sep. 21, 1998) all of these are incorporated by reference herein in their entirety.

In addition, antisense deoxyoligoribonucleotides can be used to target RNA by means of DNA-RNA interactions, thereby activating RNase H, which digests the target RNA in the duplex. Antisense DNA can be expressed via the use of a single stranded DNA intracellular expression vector or equivalents and variations thereof.

Triplex Forming Oligonucleotides (TFO): Single stranded DNA can be designed to bind to genomic DNA in a sequence specific manner. TFOs are comprised of pyrimidine-rich oligonucleotides which bind DNA helices through Hoogsteen Base-pairing (Wu-Pong, supra). The resulting triple helix composed of the DNA sense, DNA antisense, and TFO disrupts RNA synthesis by RNA polymerase. The TFO mechanism can result in gene expression or cell death since binding can be irreversible (Mukhopadhyay & Roth, supra).

2-5A Antisense Chimera: The 2-5A system is an interferon mediated mechanism for RNA degradation found in higher vertebrates (Mitra et al., 1996, Proc Nat Acad Sci USA 93, 6780-6785). Two types of enzymes, 2-5A synthetase and RNase L, are required for RNA cleavage. The 2-5A synthetases require double stranded RNA to form 2′-5′ oligoadenylates (2-5A). 2-5A then acts as an allosteric effector for utilizing RNase L which has the ability to cleave single stranded RNA. The ability to form 2-5A structures with double stranded RNA makes this system particularly useful for inhibition of viral replication.

(2′-5′) oligoadenylate structures can be covalently linked to antisense molecules to form chimeric oligonucleotides capable of RNA cleavage (Torrence, supra). These molecules putatively bind and activate a 2-5A dependent RNase, the oligonucleotide/enzyme complex then binds to a target RNA molecule which can then be cleaved by the RNase enzyme.

Enzymatic Nucleic Acid: Several varieties of naturally-occurring enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al.,1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

The enzymatic nature of an enzymatic nucleic acid molecule has significant advantages, one advantage being that the concentration of enzymatic nucleic acid molecule necessary to affect a therapeutic treatment is lower. This advantage reflects the ability of the enzymatic nucleic acid molecule to act enzymatically. Thus, a single enzymatic nucleic acid molecule is able to cleave many molecules of target RNA. In addition, the enzymatic nucleic acid molecule is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of a enzymatic nucleic acid molecule.

Nucleic acid molecules having an endonuclease enzymatic activity are able to repeatedly cleave other separate RNA molecules in a nucleotide base sequence-specific manner. With the proper design, such enzymatic nucleic acid molecules can be targeted to RNA transcripts, and achieve efficient cleavage in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving enzymatic nucleic acid molecules can be used as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology, 6, 237-250).

Enzymatic nucleic acid molecules of the invention that are allosterically regulated (“allozymes”) can be used to down-regulate EGFR expression. These allosteric enzymatic nucleic acids or allozymes (see for example George et al., U.S. Pat. Nos. 5,834,186 and 5,741,679, Shih et al., U.S. Pat. No. 5,589,332, Nathan et al., U.S. Pat. No 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842) are designed to respond to a signaling agent, for example, mutant EGFR protein, wild-type EGFR protein, mutant EGFR RNA, wild-type EGFR RNA, other proteins and/or RNAs involved in EGFR signal transduction, compounds, metals, polymers, molecules and/or drugs that are targeted to EGFR expressing cells etc., which in turn modulates the activity of the enzymatic nucleic acid molecule. In response to interaction with a predetermined signaling agent, the allosteric enzymatic nucleic acid molecule's activity is activated or inhibited such that the expression of a particular target is selectively down-regulated. The target can comprise wild-type EGFR, mutant EGFR, and/or a predetermined component of the EGFR signal transduction pathway. In a specific example, allosteric enzymatic nucleic acid molecules that are activated by interaction with a RNA encoding a mutant EGFR protein are used as therapeutic agents in vivo. The presence of RNA encoding the mutant EGFR protein activates the allosteric enzymatic nucleic acid molecule that subsequently cleaves the RNA encoding a mutant EGFR protein resulting in the inhibition of mutant EGFR protein expression. In this manner, cancerous cells that express the mutant form of the EGFR protein are selectively targeted.

In another non-limiting example, an allozyme can be activated by a EGFR protein, peptide, or mutant polypeptide that caused the allozyme to inhibit the expression of EGFR gene, by, for example, cleaving RNA encoded by EGFR gene. In this non-limiting example, the allozyme acts as a decoy to inhibit the function of EGFR and also inhibit the expression of EGFR once activated by the EGFR protein. The nucleic acid molecules of the instant invention are also referred to as GeneBloc reagents, which are essentially nucleic acid molecules (eg; ribozymes, antisense) capable of down-regulating gene expression.

Target Sites

Targets for useful enzymatic nucleic acid molecules and antisense nucleic acids can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468, and hereby incorporated by reference herein in totality. Other examples include the following PCT applications, which concern inactivation of expression of disease-related genes: WO 95/23225, WO 95/13380, WO 94/02595, incorporated by reference herein. Rather than repeat the guidance provided in those documents here, below are provided specific examples of such methods, not limiting to those in the art. Enzymatic nucleic acid molecules and antisense to such targets are designed as described in those applications and synthesized to be tested in vitro and in vivo, as also described. The sequences of human EGFR RNAs were screened for optimal enzymatic nucleic acid and antisense target sites using a computer-folding algorithm. Antisense, hammerhead, DNAzyme, NCH, amberzyme, zinzyme, or G-Cleaver enzymatic nucleic acid molecule binding/cleavage sites were identified. These sites are shown in Tables III to VII (all sequences are 5′ to 3′ in the tables; underlined regions can be any sequence “X” or linker X, the actual sequence is not relevant here). The nucleotide base position is noted in the Tables as that site to be cleaved by the designated type of enzymatic nucleic acid molecule. While human sequences can be screened and enzymatic nucleic acid molecule and/or antisense thereafter designed, as discussed in Stinchcomb et al., WO 95/23225, mouse targeted enzymatic nucleic acid molecules can be useful to test efficacy of action of the enzymatic nucleic acid molecule and/or antisense prior to testing in humans.

Antisense, hammerhead, DNAzyme, NCH, amberzyme, zinzyme or G-Cleaver enzymatic nucleic acid molecule binding/cleavage sites were identified. The nucleic acid molecules are individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions such as between the binding arms and the catalytic core are eliminated from consideration. Varying binding arm lengths can be chosen to optimize activity.

Antisense, hammerhead, DNAzyme, NCH, amberzyme, zinzyme or G-Cleaver enzymatic nucleic acid molecule binding/cleavage sites were identified and were designed to anneal to various sites in the RNA target. The binding arms are complementary to the target site sequences described above. The nucleic acid molecules were chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; and Wincott et al., 1995 Nucleic Acids Res. 23, 2677-2684; Caruthers et al., 1992, Methods in Enzymology 211, 3-19.

Synthesis of Nucleic Acid Molecules

Synthesis of nucleic acids greater than 100 nucleotides in length is difficult using automated methods, and the therapeutic cost of such molecules is prohibitive. In this invention, small nucleic acid motifs (“small refers to nucleic acid motifs less than about 100 nucleotides in length, preferably less than about 80 nucleotides in length, and more preferably less than about 50 nucleotides in length; e.g., antisense oligonucleotides, hammerhead or the NCH ribozymes) are preferably used for exogenous delivery. The simple structure of these molecules increases the ability of the nucleic acid to invade targeted regions of RNA structure. Exemplary molecules of the instant invention are chemically synthesized, and others can similarly be synthesized.

Oligonucleotides (eg; antisense GeneBlocs) are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table II outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the antisense oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder.

The method of synthesis used for normal RNA including certain enzymatic nucleic acid molecules follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1: 1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA.3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA.3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive hammerhead ribozymes or binding attenuated control (BAC) oligonucleotides) are synthesized by substituting a U for G₅ and a U for A₁₄ (numbering from Hertel, K. J., et al., 1992, Nucleic Acids Res., 20, 3252). Similarly, one or more nucleotide substitutions can be introduced in other enzymatic nucleic acid molecules to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96 well format, all that is important is the ratio of chemicals used in the reaction.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

Preferably, the nucleic acid molecules of the present invention are modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Ribozymes are purified by gel electrophoresis using general methods or are purified by high pressure liquid chromatography (HPLC; See Wincott et al., Supra, the totality of which is hereby incorporated herein by reference) and are re-suspended in water.

The sequences of the nucleic acid molecules, including enzymatic nucleic acid molecules and antisense, that are chemically synthesized, are shown in Tables III-VII. The sequences of the enzymatic nucleic acid constructs that are chemically synthesized, are complementary to the Substrate sequences shown in Tables III-VII. Those in the art will recognize that these sequences are representative only of many more such sequences where the enzymatic portion of the enzymatic nucleic acid (all but the binding arms) is altered to affect activity. The enzymatic nucleic acid construct sequences listed in Tables III-VII can be formed of ribonucleotides or other nucleotides or non-nucleotides. Such enzymatic nucleic acid molecules with enzymatic activity are equivalent to the enzymatic nucleic acid molecules described specifically in the Tables.

Optimizing Activity of the Nucleic Acid Molecule of the Invention.

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). Modifications which enhance their efficacy in cells, and removal of bases from nucleic acid molecules to shorten oligonucleotide synthesis times and reduce chemical requirements are desired. (All these publications are hereby incorporated by reference herein).

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, too many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications that maintain or enhance activity are provided. Such nucleic acid is also generally more resistant to nucleases than unmodified nucleic acid. Thus, in a cell and/or in vivo the activity may not be significantly lowered. Therapeutic nucleic acid molecules delivered exogenously are optimally stable within cells until translation of the target RNA has been inhibited long enough to reduce the levels of the undesirable protein. This period of time varies between hours to days depending upon the disease state. Clearly, nucleic acid molecules must be resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein) have expanded the ability to modify nucleic acid molecules by introducing nucleotide modifications to enhance their nuclease stability as described above.

Therapeutic nucleic acid molecules (e.g., enzymatic nucleic acid molecules and antisense nucleic acid molecules) delivered exogenously are optimally stable within cells until translation of the target RNA has been inhibited long enough to reduce the levels of the undesirable protein. This period of time varies between hours to days depending upon the disease state. These nucleic acid molecules should be resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

In another embodiment, nucleic acid catalysts having chemical modifications that maintain or enhance enzymatic activity are provided. Such nucleic acids are also generally more resistant to nucleases than unmodified nucleic acid. Thus, in a cell and/or in vivo the activity of the nucleic acid may not be significantly lowered. As exemplified herein such enzymatic nucleic acids are useful in a cell and/or in vivo even if activity over all is reduced 10 fold (Burgin et al., 1996, Biochemistry, 35, 14090). Such enzymatic nucleic acids herein are said to “maintain” the enzymatic activity of an all RNA ribozyme or all DNA DNAzyme.

In another aspect the nucleic acid molecules comprise a 5′ and/or a 3′-cap structure.

By “cap structure” is meant chemical modifications, which have been incorporated at either terminus of the oligonucleotide (see for example Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both terminus. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

In another embodiment the 3′-cap includes, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

An “alkyl” group refers to a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group can be substituted or unsubstituted. When substituted the substituted group(s) is preferably, hydroxyl, cyano, alkoxy, ═O, ═S, NO2 or N(CH3)2, amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group can be substituted or unsubstituted. When substituted the substituted group(s) is preferably, hydroxyl, cyano, alkoxy, ═O, ═S, NO2, halogen, N(CH3)2, amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group can be substituted or unsubstituted. When substituted the substituted group(s) is preferably, hydroxyl, cyano, alkoxy, ═O, ═S, NO2 or N(CH3)2, amino or SH.

Such alkyl groups can also include aryl, alkylaryl, carbocyclic aryl, heterocyclic aryl, amide and ester groups. An “aryl” group refers to an aromatic group which has at least one ring having a conjugated p electron system and includes carbocyclic aryl, heterocyclic aryl and biaryl groups, all of which can be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, for example, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

In one embodiment, the invention features modified enzymatic nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

By “abasic” is meant sugar moieties lacking a base or having other chemical groups in place of a base at the 1′ position, for example a 3′,3′-linked or 5′,5′-linked deoxyabasic ribose derivative (for more details see Wincott et al., International PCT publication No. WO 97/26270).

By “unmodified nucleoside” is meant one of the bases adenine, cytosine, guanine, thymine, uracil joined to the 1′ carbon of β-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH₂ or 2′—O— NH₂, which can be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid (e.g., antisense and ribozyme) structure can be made to enhance the utility of these molecules. For example, such modifications can enhance shelf-life, half-life in vitro, stability, and ease of introduction of such oligonucleotides to the target site, including e.g., enhancing penetration of cellular membranes and conferring the ability to recognize and bind to targeted cells.

Use of the nucleic acid-based molecules of the invention can lead to better treatment of the disease progression by affording the possibility of combination therapies (e.g., multiple enzymatic nucleic acid molecules targeted to different genes, enzymatic nucleic acid molecules coupled with known small molecule inhibitors, or intermittent treatment with combinations of enzymatic nucleic acid molecules (including different enzymatic nucleic acid molecule motifs) and/or other chemical or biological molecules). The treatment of patients with nucleic acid molecules can also include combinations of different types of nucleic acid molecules. Therapies can be devised which include a mixture of enzymatic nucleic acid molecules (including different enzymatic nucleic acid molecule motifs), antisense and/or 2-5A chimera molecules to one or more targets to alleviate symptoms of a disease.

Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example though the use of conjugates and biodegradable polymers. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. NeuroVirol., 3, 387-400. More detailed descriptions of nucleic acid delivery and administration are provided in Sullivan et al., supra, Draper et al., PCT WO93/23569, Beigelman et al., PCT WO99/05094, and Klimuk et al., PCT WO99/04819 all of which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, DF et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al.,1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a patient in combination with other therapeutic compounds to increase the overall therapeutic effect. The use of multiple compounds to treat an indication can increase the beneficial effects while reducing the presence of side effects.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totalities by reference herein). Gene therapy approaches specific to the CNS are described by Blesch et al., 2000, Drug News Perspect., 13, 269-280; Peterson et al., 2000, Cent. Nerv. Syst. Dis., 485-508; Peel and Klein, 2000, J. Neurosci. Methods, 98, 95-104; Hagihara et al., 2000, Gene Ther., 7, 759-763; and Herrlinger et al., 2000, Methods Mol. Med., 35, 287-312. AAV-mediated delivery of nucleic acid to cells of the nervous system is further described by Kaplitt et al., U.S. Pat. No. 6,180,613.

In another aspect of the invention, RNA molecules of the present invention are preferably expressed from transcription units (see for example Couture et al., 1996, TIG., 12, 510) inserted into DNA or RNA vectors. The recombinant vectors are preferably DNA plasmids or viral vectors. Ribozyme expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intra-muscular administration, by administration to target cells ex-planted from the patient followed by reintroduction into the patient, or by any other means that would allow for introduction into the desired target cell (for a review see Couture et al., 1996, TIG., 12, 510).

In one aspect the invention features an expression vector comprising a nucleic acid sequence encoding at least one of the nucleic acid molecules of the instant invention is disclosed. The nucleic acid sequence encoding the nucleic acid molecule of the instant invention is operable linked in a manner which allows expression of that nucleic acid molecule.

In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector can optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. U S A, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. U S A, 89, 10802-6; Chen et al., 1992, Nucleic Acids Res., 20, 4581-9; Yu et al., 1993, Proc. Natl. Acad. Sci. USA, 90, 6340-4; L'Huillier et al., 1992, EMBO J., 11, 4411-8; Lisziewicz et al., 1993, Proc. Natl. Acad. Sci. U. S. A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

In another embodiment the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES.

The following are non-limiting examples showing the selection, isolation, synthesis and activity of nucleic acids of the instant invention.

The following examples demonstrate the selection and design of Antisense, hammerhead, DNAzyme, NCH, Amberzyme, Zinzyme, or G-Cleaver ribozyme molecules and binding/cleavage sites within EGFR RNA.

### Example 1

Identification of Potential Target Sites in Human EGFR RNA

The sequence of human EGFR genes are screened for accessible sites using a computer-folding algorithm. Regions of the RNA that do not form secondary folding structures and contained potential enzymatic nucleic acid molecule and/or antisense binding/cleavage sites are identified. The sequences of these binding/cleavage sites are shown in Tables III-VII. Sequences shown in Tables III-VII are RNA sequences that are homologous to HER1, HER2, HER3, and HER4 genes.

### Example 2

Selection of Enzymatic Nucleic Acid Cleavage Sites in Human EGFR RNA

Enzymatic nucleic acid molecule target sites are chosen by analyzing sequences of Human EGFR genes HER1, HER2, HER3, and HER4 (Genbank accession No: NM_{—}005228, NM_{—}004448, NM_{—}001982, and NM_{—}005235 respectively) and prioritizing the sites on the basis of folding. Enzymatic nucleic acid molecules are designed that can bind each target and are individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the enzymatic nucleic acid molecule sequences fold into the appropriate secondary structure. Those enzymatic nucleic acid molecules with unfavorable intramolecular interactions between the binding arms and the catalytic core are eliminated from consideration. As noted below, varying binding arm lengths can be chosen to optimize activity. Generally, at least 4 bases on each arm are able to bind to, or otherwise interact with, the target RNA.

### Example 3

Chemical Synthesis and Purification of Ribozymes and Antisense for Efficient Cleavage and/or Blocking of EGFR RNA

Enzymatic nucleic acid molecules and antisense constructs are designed to anneal to various sites in the RNA message. The binding arms of the enzymatic nucleic acid molecules are complementary to the target site sequences described above, while the antisense constructs are fully complementary to the target site sequences described above. The enzymatic nucleic acid molecules and antisense constructs were chemically synthesized. The method of synthesis used followed the procedure for normal RNA synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were typically >98%.

Enzymatic nucleic acid molecules and antisense constructs are also synthesized from DNA templates using bacteriophage T7 RNA polymerase (Milligan and Uhlenbeck, 1989, Methods Enzymol. 180, 51). Enzymatic nucleic acid molecules and antisense constructs are purified by gel electrophoresis using general methods or are purified by high pressure liquid chromatography (HPLC; See Wincott et al., supra; the totality of which is hereby incorporated herein by reference) and are resuspended in water. The sequences of the chemically synthesized enzymatic nucleic acid molecules used in this study are shown below in Table III-VII. The sequences of the chemically synthesized antisense constructs used in this study are complementary sequences to the Substrate sequences shown below as in Table III-VII.

### Example 4

Enzymatic Nucleic Acid Molecule Cleavage of EGFR RNA Target in vitro

Enzymatic nucleic acid molecules targeted to the human EGFR RNA are designed and synthesized as described above. These enzymatic nucleic acid molecules can be tested for cleavage activity in vitro, for example, using the following procedure. The target sequences and the nucleotide location within the EGFR RNA are given in Tables III-VII.

Cleavage Reactions: Full-length or partially full-length, internally-labeled target RNA for enzymatic nucleic acid molecule cleavage assay is prepared by in vitro transcription in the presence of [a-³²p] CTP, passed over a G 50 Sephadex column by spin chromatography and used as substrate RNA without further purification. Alternately, substrates are 5′-³²P-end labeled using T4 polynucleotide kinase enzyme. Assays are performed by pre-warming a 2×concentration of purified enzymatic nucleic acid molecule in enzymatic nucleic acid molecule cleavage buffer (50 mM Tris-HCl, pH 7.5 at 37° C., 10 mM MgCl₂) and the cleavage reaction was initiated by adding the 2×enzymatic nucleic acid molecule mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM enzymatic nucleic acid molecule, i.e., enzymatic nucleic acid molecule excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by enzymatic nucleic acid molecule cleavage are visualized on an autoradiograph of the gel. The percentage of cleavage is determined by Phosphor Imager® quantitation of bands representing the intact substrate and the cleavage products.

### Example 5

In vivo Models Used to Evaluate the Down-regulation of EGFR Gene Expression

Nucleic acid molecules targeted to the human EGFR RNA are designed and synthesized as described above. These nucleic acid molecules can be tested for cleavage activity in vivo, for example, using the procedures described below.A variety of endpoints have been used in cell culture models to evaluate EGFR-mediated effects after treatment with anti-EGFR agents. Phenotypic endpoints include inhibition of cell proliferation, apoptosis assays and reduction of EGFR protein expression. Because overexpression of EGFR is directly associated with increased proliferation of tumor cells, a proliferation endpoint for cell culture assays is preferably used as a primary screen. There are several methods by which this endpoint can be measured. Following treatment of cells with nucleic acid molecules, cells are allowed to grow (typically 5 days) after which either the cell viability, the incorporation of [³H] thymidine into cellular DNA and/or the cell density can be measured. The assay of cell density is well-known to those skilled in the artand can, for example, be performed in a 96-well format using commercially available fluorescent nucleic acid stains (such as Syto® 13 or CyQuant®). For example, the assay using CyQuant® is described herein.

As a secondary, confirmatory endpoint, a nucleic acid-mediated decrease in the level of EGFR RNA and/or EGFR protein expression can be evaluated using methods known in the art, such as RT-PCR, Northern blot, ELISA, Western blot, and immunoprecipitation analyses, to name a few techniques.

Validation of Cell Lines and Ribozyme Treatment Conditions

Two human cell lines (A549 and SKOV-3) that are known to express medium to high levels of EGFR protein are considered for nucleic acid screening. In order to validate these cell lines for EGFR-mediated sensitivity, both cell lines are treated with an EGFR specific antibody, for example mAB IMC-C225 (InClone) and its effect on cell proliferation is determined. mAB is added to cells at concentrations ranging from 0-8 μM in medium containing either no serum (OptiMem), 0.1% or 0.5% FBS and efficacy is determined via cell proliferation. Inhibition of proliferation (˜50%) in both cell lines after addition of mAB at 0.5 nM in medium containing 0.1% or no FBS, indicates that both cell lines are sensitive to an anti-EGFR agent (mAB) and supports their use in experiments testing anti-EGFR nucleic acid molecules.

Prior to nucleic acid screening, the choice of the optimal lipid(s) and conditions for nucleic acid delivery is determined empirically for each cell line. Applicant has established a panel of cationic lipids (lipids as described in PCT application WO99/05094) that can be used to deliver nucleic acids to cultured cells and are useful for cell proliferation assays that are typically 3-5 days in length. Additional description of useful lipids is provided above, and those skilled in the art are also familiar with a variety of lipids that can be used for delivery of oligonucleotide to cells in culture. Initially, this panel of lipid delivery vehicles is screened in A549 and SKOV-3 cells using previously established control oligonucleotides. Specific lipids and conditions for optimal delivery are selected for each cell line based on these screens. These conditions are used to deliver EGFR specific nucleic acids to cells for primary (inhibition of cell proliferation) and secondary (decrease in EGFR RNA/protein) efficacy endpoints.

Primary Screen: Inhibition of Cell Proliferation

Nucleic acid screens were performed using an automated, high throughput 96-well cell proliferation assay. Cell proliferation was measured over a 5-day treatment period using the nucleic acid stain CyQuant® for determining cell density. The growth of cells treated with enzymatic nucleic acid/lipid complexes were compared to both untreated cells and to cells treated with Scrambled-arm Attenuated core Controls (SAC). SACs can no longer bind to the target site due to the scrambled arm sequence and have nucleotide changes in the core that greatly diminish nucleic acid cleavage. These SACs are used to determine non-specific inhibition of cell growth caused by nucleic acid chemistry (i.e. multiple 2′ O-Me modified nucleotides, a single 2′C-allyl uridine, 4 phosphorothioates and a 3′ inverted a basic). The growth of cells treated with GeneBloc/lipid complexes were compared to both untreated cells and to cells treated with a scrambled control GeneBloc that can no longer bind to the target site due to the scrambled sequence. Lead nucleic acids are chosen from the primary screen based on their ability to inhibit cell proliferation in a specific manner. Dose response assays are carried out on these leads and a subset was advanced into a secondary screen using the level of EGFR protein as an endpoint.

Secondary Screen: Decrease in EGER Protein and/or RNA

A secondary screen that measures the effect of anti-EGFR nucleic acids on EGFR protein and/or RNA levels is used to affirm preliminary findings. A EGFR ELISA for both A549 and SKOV-3 cells can been established and made available for use as an additional endpoint. In addition, a real time RT-PCR assay (TaqMan assay) has been developed to assess EGFR RNA reduction. Dose response activity of nucleic acid molecules of the instant invention can be used to assess both EGFR protein and RNA reduction endpoints.

Enzymatic Nucleic Acid Mechanism Assays

A TaqMan® assay for measuring the enzymatic nucleic acid-mediated decrease in EGFR RNA has been established. This assay is based on PCR technology and can measure in real time the production of EGFR mRNA relative to a standard cellular mRNA such as GAPDH. This RNA assay is used to establish proof that lead enzymatic nucleic acids are working through an RNA cleavage mechanism and result in a decrease in the level of EGFR mRNA, thus leading to a decrease in cell surface EGFR protein receptors and a subsequent decrease in tumor cell proliferation.

Animal Models

Evaluating the efficacy of anti-EGFR agents in animal models is an important prerequisite to human clinical trials. As in cell culture models, the most EGFR sensitive mouse tumor xenografts are those derived from human carcinoma cells that express high levels of EGFR protein. In a recent study, nude mice bearing human vulvar (A431), lung (A549 and SK-LC-16 NSCL and LX-1) and prostate (PC-3 and TSU-PRI) xenografts were sensitive to the anti-EGFR tyrosine kinase inhibitor ZD1839 (Iressa), resulting in a partial regression of A431 tumor growth, 70-80% inhibition of tumor growth (A549, SKLC-16, TSU-PRI and PC-3 tumors), and 50-55% inhibition against the LX-1 tumor at a 150 mg kg dose (ip, every 3-4 days×4), (Sirotnak et al., 2000, Clin. Cancer Res., 6, 4885-48892). This same study compared the efficacy of ZD1839 alone or in combination with the commonly used chemotherapeutics, cisplatin, carboplatin, paclitaxel, docetaxel, edatrexate, gemcitabine, vinorelbine. When used in combination with certain chemotherapeutic agents, most notably cisplatin, carboplatin, paclitaxel, docetaxel, and edatrexate, marked response was observed compared to treatment with these agents alone, resulting in partial or complete regression in some cases. The above studies provide evidence that inhibition of EGFR expression by anti-EGFR agents causes inhibition of tumor growth in animals.

Animal Model Development

Tumor cell lines (A549 and SKOV-3) are characterized to establish their growth curves in mice. These cell lines are implanted into both nude and SCID mice and primary tumor volumes are measured 3 times per week. Growth characteristics of these tumor lines using a Matrigel implantation format can also be established. The use of other cell lines that have been engineered to express high levels of EGFR can also be used in the described studies. The tumor cell line(s) and implantation method that supports the most consistent and reliable tumor growth is used in animal studies testing the lead EGFR nucleic acid(s). Nucleic acids are administered by daily subcutaneous injection or by continuous subcutaneous infusion from Alzet mini osmotic pumps beginning 3 days after tumor implantation and continuing for the duration of the study. Group sizes of at least 10 animals are employed. Efficacy is determined by statistical comparison of tumor volume of nucleic acid-treated animals to a control group of animals treated with saline alone. Because the growth of these tumors is generally slow (45-60 days), an initial endpoint is the time in days it takes to establish an easily measurable primary tumor (i.e. 50-100 mm³) in the presence or absence of nucleic acid treatment.

EGFR Protein Levels for Patient Screening and as a Potential Endpoint

Because elevated EGFR levels can be detected in several cancers, cancer patients can be pre-screened for elevated EGFR prior to admission to initial clinical trials testing an anti-EGFR nucleic acid. Initial EGFR levels can be determined (by ELISA) from tumor biopsies or resected tumor samples. During clinical trials, it may be possible to monitor circulating EGFR protein by ELISA. Evaluation of serial blood/serum samples over the course of the anti-EGFR nucleic acid treatment period could be useful in determining early indications of efficacy.

### Example 7

Activity of Nucleic Acid Molecules Used to Down-regulate EGFR Gene Expression

Applicant has designed, synthesized and tested several nucleic acid molecules targeted against EGFR RNA in cell proliferation and RNA reduction assays described herein.

Proliferation Assay

The model proliferation assay used in the study requires a cell-plating density of 2,000-10,000 cells/well in 96-well plates and at least 2 cell doublings over a 5-day treatment period. Cells used in proliferation studies were either lung or ovarian cancer cells (A549 and SKOV-3 cells respectively). To calculate cell density for proliferation assays, the FIPS (fluoro-imaging processing system) method known in the art was used. This method allows for cell density measurements after nucleic acids are stained with CyQuant® dye, and has the advantage of accurately measuring cell densities over a very wide range 1,000-100,000 cells/well in 96-well format.

### Example 8

Activity of Nucleic Acid Molecules Used to Down-regulate EGFR and HER2 Gene Expression

Applicant has designed nucleic acid molecules that target all four members of the EGFR family (HER1, HER2, HER3, and HER4). These nucleic acid molecules can be tested in cell proliferation and RNA reduction assays described herein. The use a single nucleic acid molecule that can target HER1, HER2, HER3, and HER4 RNA in a sequence specific manner can be advantageous in inhibiting the expression of tyrosine kinase proteins that are up-regulated in a variety of cancers. Furthermore, Brandt et al., 1999, FASEB. J., 13, 1939-1949, propose that HER2 and EGFR are dominant heterodimer partners that determine a motogenic phenotype in human breast cancer cells. The use of nucleic acid molecules that target HER1, HER2, HER3, and HER4 RNA is advantageous since only one composition is used to inhibit both targets and can potentially provide a synergistic or additive therapeutic effect.

Indications

The present body of knowledge in EGFR research indicates the need for methods to assay EGFR activity and for compounds that can regulate EGFR expression for research, diagnostic, and therapeutic use. As described herein, the nucleic acid molecules of the present invention can be used in assays to diagnose disease state related of EGF-F levels. In addition, the nucleic acid molecules can be used to treat disease state related to EGF-R levels.

Particular degenerative and disease states that can be associated with EGFR level include, but are not limited to, cancers and cancerous conditions such as breast, lung, prostate, colorectal, brain, esophageal, stomach, bladder, pancreatic, cervical, head and neck, and ovarian cancer, melanoma, lymphoma, glioma, multidrug resistant cancers, and any other diseases or conditions that are related to or will respond to the levels of EGFR in a cell or tissue, alone or in combination with other therapies.

The use of monoclonal antibodies (eg; mAb INC C225, mAB ABX-EGF) treatment, EGFR-specific tyrosine kinase inhibitors (TKIs), for example OSI-774 and ZD1839, chemotherapy, and/or radiation therapy, are all non-limiting examples of a methods that can be combined with or used in conjunction with the nucleic acid molecules (e.g. ribozymes and antisense molecules) of the instant invention. Common chemotherapies that can be combined with nucleic acid molecules of the instant invention include various combinations of cytotoxic drugs to kill the cancer cells. These drugs include but are not limited to paclitaxel (Taxol), docetaxel, cisplatin, methotrexate, cyclophosphamide, doxorubin, fluorouracil carboplatin, edatrexate, gemcitabine, vinorelbine etc. Those skilled in the art will recognize that other drug compounds and therapies can be similarly be readily combined with the nucleic acid molecules of the instant invention (e.g. ribozymes and antisense molecules) are hence within the scope of the instant invention.

Diagnostic Uses

The nucleic acid molecules of this invention (e.g., enzymatic nucleic acid molecules) can be used as diagnostic tools to examine genetic drift and mutations within diseased cells or to detect the presence of EGFR RNA in a cell. The close relationship between enzymatic nucleic acid molecule activity and the structure of the target RNA allows the detection of mutations in any region of the molecule which alters the base-pairing and three-dimensional structure of the target RNA. By using multiple enzymatic nucleic acid molecules described in this invention, one can map nucleotide changes which are important to RNA structure and function in vitro, as well as in cells and tissues. Cleavage of target RNAs with enzymatic nucleic acid molecules can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g., multiple enzymatic nucleic acid molecules targeted to different genes, enzymatic nucleic acid molecules coupled with known small molecule inhibitors, or intermittent treatment with combinations of enzymatic nucleic acid molecules and/or other chemical or biological molecules). Other in vitro uses of enzymatic nucleic acid molecules of this invention are well known in the art, and include detection of the presence of mRNAs associated with EGFR-related condition. Such RNA is detected by determining the presence of a cleavage product after treatment with an enzymatic nucleic acid molecule using standard methodology.

In a specific example, enzymatic nucleic acid molecules which cleave only wild-type or mutant forms of the target RNA are used for the assay. The first enzymatic nucleic acid molecule is used to identify wild-type RNA present in the sample and the second enzymatic nucleic acid molecule is used to identify mutant RNA in the sample. As reaction controls, synthetic substrates of both wild-type and mutant RNA are cleaved by both enzymatic nucleic acid molecules to demonstrate the relative enzymatic nucleic acid molecule efficiencies in the reactions and the absence of cleavage of the “non-targeted” RNA species. The cleavage products from the synthetic substrates also serve to generate size markers for the analysis of wild-type and mutant RNAs in the sample population. Thus each analysis requires two enzymatic nucleic acid molecules, two substrates and one unknown sample which is combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., EGFR) is adequate to establish risk. If probes of comparable specific activity are used for both transcripts, then a qualitative comparison of RNA levels will be adequate and will decrease the cost of the initial diagnosis. Higher mutant form to wild-type ratios are correlated with higher risk whether RNA levels are compared qualitatively or quantitatively. The use of enzymatic nucleic acid molecules in diagnostic applications contemplated by the instant invention is described, for example, in George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842.

Additional Uses

Potential uses of sequence-specific enzymatic nucleic acid molecules of the instant invention can have many of the same applications for the study of RNA that DNA restriction endonucleases have for the study of DNA (Nathans et al., 1975 Ann. Rev. Biochem. 44:273). For example, the pattern of restriction fragments can be used to establish sequence relationships between two related RNAs, and large RNAs can be specifically cleaved to fragments of a size more useful for study. The ability to engineer sequence specificity of the enzymatic nucleic acid molecule is ideal for cleavage of RNAs of unknown sequence. Applicant has described the use of nucleic acid molecules to down-regulate gene expression of target genes in bacterial, microbial, fungal, viral, and eukaryotic systems including plant, or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the following claims.

TABLE I
Characteristics of naturally occurring ribozymes

Group I Introns
Size: ˜150 to >1000 nucleotides.
Requires a U in the target sequence immediately 5′ of the cleavage site.
Binds 4-6 nucleotides at the 5′-side of the cleavage site.
Reaction mechanism: attack by the 3′-OH of guanosine to generate
cleavage products with 3′-OH and 5′-guanosine.
Additional protein cofactors required in some cases to help folding and
maintenance of the active structure.
Over 300 known members of this class. Found as an intervening
sequence in Tetrahymena thermophila rRNA, fungal
mitochondria, chloroplasts, phage T4, blue-green algae, and others.
Major structural features largely established through phylogenetic
comparisons, mutagenesis, and biochemical studies^{i, ii}.
Complete kinetic framework established for one ribozyme^{iii, iv, v, vi}.
Studies of ribozyme folding and substrate docking underway^{vii, viii, ix}.
Chemical modification investigation of important residues well
established^{x, xi}.
The small (4-6 nt) binding site may make this ribozyme too non-specific
for targeted RNA cleavage, however, the Tetrahymena group I intron
has been used to repair a “defective” β-galactosidase message
by the ligation of new galactosidase sequences onto the defective
message^{xii}.
RNAse P RNA (M1 RNA)
Size: ˜290 to 400 nucleotides.
RNA portion of a ubiquitous ribonucleoprotein enzyme.
Cleaves tRNA precursors to form mature tRNA^{xiii}.
Reaction mechanism: possible attack by M²⁺-OH to generate cleavage
products with 3′-OH and 5′-phosphate.
RNAse P is found throughout the prokaryotes and eukaryotes.
The RNA subunit has been sequenced from bacteria, yeast, rodents,
and primates.
Recruitment of endogenous RNAse P for therapeutic applications is
possible through hybridization of an External Guide Sequence
(EGS) to the target RNA^{xiv, xvi}
Important phosphate and 2′ OH contacts recently identified^{xvi, xvii}
Group II Introns
Size: >1000 nucleotides.
Trans cleavage of target RNAs recently demonstrated^{xviii, xix}.
Sequence requirements not fully determined.
Reaction mechanism: 2′-OH of an internal adenosine generates
cleavage products with 3′-OH and a “lariat” RNA
containing a 3′-5′ and a 2′-5′ branch point.
Only natural ribozyme with demonstrated participation in DNA
cleavage^{xx, xxi} in addition to RNA cleavage and ligation.
Major structural features largely established through phylogenetic
comparisons^{xxii}
Important 2′ OH contacts beginning to be identified^{xxiii}
Kinetic framework under development^{xxiv}
Neurospora VS RNA
Size: ˜144 nucleotides.
Trans cleavage of hairpin target RNAs recently demonstrated^{xxvll .}
Sequence requirements not fully determined.
Reaction mechanism: attack by 2′-OH 5′ to the scissile bond to
generate cleavage products with 2′, 3′-cyclic phosphate and 5′-OH ends,
Binding sites and structural requirements not fully determined.
Only 1 known member of this class. Found in Neurospora VS RNA.
Hammerhead Ribozyme
(see text for references)
Size: ˜13 to 40 nucleotides.
Requires the target sequence UH immediately 5′ of the cleavage site.
Binds a variable number nucleotides on both sides of the cleavage site.
Reaction mechanism: attack by 2′-OH 5′ to the scissile bond to
generate cleavage products with 2′, 3′-cyclic phosphate and 5′-OH ends.
14 known members of this class. Found in a number of plant pathogens
(virusoids) that use RNA as the infectious agent.
Essential structural features largely defined, including 2 crystal
structures^{xxvi},^{xxvii}
Minimal ligation activity demonstrated (for engineering through in vitro
selection)^{xxviii}
Complete kinetic framework established for two or more ribozymes^{xxix}.
Chemical modification investigation of important residues well
established^{xxx}.
Hairpin Ribozyme
Size: ˜50 nucleotides.
Requires the target sequence GUC immediately 3′ of the cleavage site.
Binds 4-6 nucleotides at the 5′-side of the cleavage site and a variable
number to the 3′-side of the cleavage site.
Reaction mechanism: attack by 2′-OH 5′to the scissile bond to generate
cleavage products with 2′, 3′-cyclic phosphate and 5′-OH ends.
3 known members of this class. Found in three plant pathogen (satellite
RNAs of the tobacco ringspot virus, arabis mosaic virus and chicory
yellow mottle virus) which uses RNA as the infectious agent.
Essential structural features largely defined^{xxxi, xxxii, xxxiii, xxxiv}
Ligation activity (in addition to cleavage activity) makes ribozyme
amenable to engineering through in vitro selection^{xxxv}
Complete kinetic framework established for one ribozyme^{xxxvi}.
Chemical modification investigation of important residues
begun^{xxxvii, xxxviii}
Hepatitis Delta Virus (HDV) Ribozyme
Size: ˜60 nucleotides.
Trans cleavage of target RNAs demonstrated^{xxxix}.
Binding sites and structural requirements not fully determined, although no
sequences 5′ of cleavage site are required. Folded ribozyme contains
a pseudoknot structure^{xl}
Reaction mechanism: attack by 2′-OH 5′to the scissile bond to
generate cleavage products with 2′, 3′-cyclic phosphate and 5′-OH ends.
Only 2 known members of this class. Found in human HDV.
Circular form of HDV is active and shows increased nuclease stability^{xli}
ⁱMichel, Francois; Westhof, Eric. Slippery substrates. Nat. Struct. Biol. (1994), 1(1), 5-7.
ⁱⁱLisacek, Frederique; Diaz, Yolande; Michel, Francois. Automatic identification of group I intron
#cores in genomic DNA sequences. J. Mol. Biol. (1994), 235(4), 1206-17.
ⁱⁱⁱHerschlag, Daniel; Cech, Thomas R.. Catalysis of RNA cleavage by the Tetrahymena thermophila
#ribozyme. 1. Kinetic description of the reaction of an RNA substrate complementary to the active site.
#Biochemistry (1990), 29(44), 10159-71.
^{iv}Herschlag, Daniel; Cech, Thomas R.. Catalysis of RNA cleavage by the Tetrahymena thermophila
#ribozyme. 2. Kinetic description of the reaction of an RNA substrate that forms a mismatch at the active
#site. Biochemistry (1990), 29(44), 10172-80.
^{v}Knitt, Deborah S.; Herschlag, Daniel. pH Dependencies of the Tetrahymena Ribozyme Reveal an
#Unconventional Origin of an Apparent pKa. Biochemistry (1996), 35(5), 1560-70.
^{vi}Bevilacqua, Philip C.; Sugimoto, Naoki; Turner, Douglas H.. A mechanistic framework for the
#second step of splicing catalyzed by the Tetrahymena ribozyme. Biochemistry (1996), 35(2), 648-58.
^{vii}Li, Yi; Bevilacqua, Philip C.; Mathews, David; Turner, Douglas H.. Thermodynamic and
#activation parameters for binding of a pyrene-labeled substrate by the Tetrahymena ribozyme: docking is
#not diffusion-controlled and is driven by a favorable entropy change. Biochemistry (1995), 34(44), 14394-9.
^{viii}Banerjee, Aloke Raj; Turner, Douglas H.. The time dependence of chemical modification reveals
#slow steps in the folding of a group I ribozyme. Biochemistry (1995), 34(19), 6504-12.
^{ix}Zarrinkar, Patrick P.; Williamson, James R.. The P9.1-P9.2 peripheral extension helps guide
#folding of the Tetrahymena ribozyme. Nucleic Acids Res. (1996), 24(5), 854-8.
^{x}Strobel, Scott A.; Cech, Thomas R.. Minor groove recognition of the conserved G.cntdot.U pair at
#the Tetrahymena ribozyme reaction site. Science (Washington, D. C.) (1995), 267(5198), 675-9.
^{xi}Strobel, Scott A.; Cech, Thomas R.. Exocyclc Amine of the Conserved G.cntdot.U Pair at the
#Cleavage Site of the Tetrahymena Ribozyme Contributes to 5′-Splice Site Selection and Transition State
#Stabilization. Biochemistry (1996), 35(4), 1201-11.
^{xii}Sullenger, Bruce A.; Cech, Thomas R.. Ribozyme-mediated repair of defective mRNA by targeted
#trans-splicing. Nature (London) (1994), 371 (6498), 619-22.
^{xiii}Robertson, HD.; Altman, S.; Smith, J.D. J. Boil. Chem, 247, 5243-5251 (1972).
^{xiv}Forster, Anthony C.; Altman, Sidney. External guide sequences for an RNA enzyme. Science
#(Washington, D. C., 1883-) (1990), 249(4970), 783-6.
^{xv}Yuan, Y.; Hwang, E. S.; Altman, S. Targeted cleavage of mRNA by human RNase P. Proc. Natl.
#Acad. Sci. USA (1992) 89, 8006-10.
^{xvi}Harris, Michael E.; Pace, Norman R.. Identification of phosphates involved in catalysis by the
#ribozyme RNase P RNA. RNA (1995), 1(2), 210-18.
^{xvii}Pan, Tao; Loria, Andrew; Zhong, Kun. Probing of tertiary interactions in RNA: 2′hydroxyl-base
#contacts between the RNase P RNA and pre-tRNA. Proc. Natl. Acad. Sci. U.S.A. (1995), 92(26), 12510-14.
^{xviii}Pyle, Anna Marie; Green, Justin B.. Building a Kinetic Framework for Group II Intron Ribozyme
#Activity: Quantitation of Interdomain Binding and Reaction Rate. Biochemistry (1994), 33(9), 2716-25.
^{xix}Michels, William J. Jr.; Pyle, Anna Marie. Conversion of a Group II Intron into a New Multiple-
#Turnover Ribozyme that Selectively Cleaves Oligonucleotides: Elucidation of Reaction Mechanism and
#Structure/Function Relationships. Biochemistry (1995), 34(9), 2965-77.
^{xx}Zimmerly, Steven; Guo, Huatao; Eskes, Robert; Yang, Jian; Perlman, Philip S.; Lambowitz, Alan
#M.. A group II intron RNA is a catalytic component of a DNA endonuclease involved in intron mobility.
#Cell (Cambridge, Mass.) (1995), 83(4), 529-38.
^{xxi}Griffin, Edmund A., Jr.; Qin, Zhifeng; Michels, Williams J., Jr.; Pyle, Anna Marie. Group II intron
#ribozymes that cleave DNA and RNA linkages with similar efficiency, and lack contacts with substrate 2′-
#hydroxyl groups. Chem. Biol. (1995), 2(11), 761-70.
^{xxii}Michel, Francois; Ferat, Jean Luc. Structure and activities of group II introns. Annu. Rev.
#Biochem. (1995), 64,435-61.
^{xxiii}Abramovitz, Dana L.; Friedman, Richard A.; Pyle, Anna Marie. Catalytic role of 2′-hydroxyl
#groups within a group II intron active site. Science (Washington, D. C.) (1996), 271(5254), 1410-13.
^{xxiv}Daniels, Danette L.; Michels, William J., Jr.; Pyle, Anna Marie. Two competing pathways for self-
#splicing by group II introns: a quantitative analysis of in vitro reaction rates and products. J. Mol. Biol.
#(1996), 256(1), 31-49.
^{xxv}Guo, Hans C. T.; Collins, Richard A.. Efficient trans-cleavage of a stem-loop RNA substrate by a
#ribozyme derived from Neurospora VS RNA. EMBO J. (1995), 14(2), 368-76.
^{xxvi}Scott, W. G., Finch, J. T., Aaron, K. The crystal structure of an all RNA hammerhead
#ribozyme: Aproposed mechanism for RNA catalytic cleavage. Cell, (1995), 81, 991-1002.
^{xxvii}McKay, Structure and function of the hammerhead ribozyme: an unfinished story. RNA, (1996),
#2, 395-403.
^{xxviii}Long, D., Uhlenbeck, O., Hertel, K. Ligation with hammerhead ribozymes. U.S. Pat. No.
#5,633,133.
^{xxix}Hertel, K. J., Herschlag, D., Uhlenbeck, O. A kinetic and thermodynamic framework for the
#hammerhead ribozyme reaction. Biochemistry, (1994) 33, 3374-3385. Beigelman, L., et al., Chemical
#modifications of hammerhead ribozymes. J. Biol. Chem., (1995) 270, 25702-25708.
^{xxx}Beigelman, L., et al., Chemical modifications of hammerhead ribozymes. J. Biol. Chem., (1995)
#270, 25702-25708.
^{xxxi}Hampel, Arnold; Tritz, Richard; Hicks, Margaret; Cruz, Phillip. ‘Hairpin’ catalytic RNA model:
#evidence for helixes and sequence requirement for substrate RNA. Nucleic Acids Res. (1990), 18(2), 299-304.
^{xxxii}Chowrira, Bharat M.; Berzal-Herranz, Alfredo; Burke, John M.. Novel guanosine requirement for
#catalysis by the hairpin ribozyme. Nature (London) (1991), 354(6351), 320-2.
^{xxxiii}Berzal-Herranz, Alfredo; Joseph, Simpson; Chowrira, Bharat M.; Butcher, Samuel E.; Burke, John
#M.. Essential nucleotide sequences and secondary structure elements of the hairpin ribozyme. EMBO J.
#(1993), 12(6), 2567-73.
^{xxxiv}Joseph, Simpson; Berzal-Herranz, Alfredo; Chowrira, Bharat M.; Butcher, Samuel E.. Substrate
#selection rules for the hairpin ribozyme determined by in vitro selection, mutation, and analysis of
#mismatched substrates. Genes Dev. (1993), 7(1), 130-8.
^{xxxv}Berzal-Herranz, Alfredo; Joseph, Simpson; Burke, John M.. In vitro selection of active hairpin
#ribozymes by sequential RNA-catalyzed cleavage and ligation reactions. Genes Dev. (1992), 6(1), 129-34.
^{xxxvi}Hegg, Lisa A.; Fedor, Martha J.. Kinetics and Thermodynamics of Intermolecular Catalysis by
#Hairpin Ribozymes. Biochemistry (1995), 34(48), 15813-28.
^{xxxvii}Grasby, Jane A.; Mersmann, Karin; Singh, Mohinder; Gait, Michael J.. Purine Functional Groups
#in Essential Residues of the Hairpin Ribozyme Required for Catalytic Cleavage of RNA. Biochemistry
#(1995), 34(12), 4068-76.
^{xxxviii}Schmidt, Sabine; Beigelman, Leonid; Karpeisky, Alexander; Usman, Nassim; Sorensen, Ulrik S.;
#Gait, Michael J.. Base and sugar requirements for RNA cleavage of essential nucleoside residues in
#internal loop B of the hairpin ribozyme: implications for secondary structure. Nucleic Acids Res. (1996),
#24(4), 573-81.
^{xxxix}Perrotta, Anne T.; Been, Michael D.. Cleavage of oligoribonucleotides by a ribozyme derived from
#the hepatitis .delta. virus RNA sequence. Biochemistry (1992), 31(1), 16-21.
^{xl}Perrotta, Anne T.; Been, Michael D.. A pseudoknot-like structure required for efficient self-
#cleavage of hepatitis delta virus RNA. Nature (London) (1991), 350(6317), 434-6.
^{xli}Puttaraju, M.; Perrotta, Anne T.; Been, Michael D.. A circular trans-acting hepatitis delta virus
#ribozyme. Nucleic Acids Res. (1993), 21(18), 4253-8.

TABLE II

| | | | | | |
|---|---|---|---|---|---|
| Reagent | Equivalents | Amount | Wait Time* DNA | Wait Time* 2′-O-methyl | Wait Time*RNA |

A. 2.5 μmol Synthesis Cycle ABI 394 Instrument

| | | | | | | | | | |
|---|---|---|---|---|---|---|---|---|---|
| Phosphoramidites | 6.5 | 163 | μL | 45 | sec | 2.5 | min | 7.5 | min |
| S-Ethyl Tetrazole | 23.8 | 238 | μL | 45 | sec | 2.5 | min | 7.5 | min |
| Acetic Anhydride | 100 | 233 | μL | 5 | sec | 5 | sec | 5 | sec |
| N-Methyl | 186 | 233 | μL | 5 | sec | 5 | sec | 5 | sec |
| Imidazole | | | | | | | | | |
| TCA | 176 | 2.3 | mL | 21 | sec | 21 | sec | 21 | sec |
| Iodine | 11.2 | 1.7 | mL | 45 | sec | 45 | sec | 45 | sec |
| Beaucage | 12.9 | 645 | μL | 100 | sec | 300 | sec | 300 | sec |
| Acetonitrile | NA | 6.67 | mL | NA | | NA | | NA | |

| | | | | | |
|---|---|---|---|---|---|
| Reagent | Equivalents | Amount | Wait Time* DNA | Wait Time* 2′-O-methyl | Wait Time*RNA |

B. 0.2 μmol Synthesis Cycle ABI 394 Instrument

| | | | | | | | | | |
|---|---|---|---|---|---|---|---|---|---|
| Phosphoramidites | 15 | 31 | μL | 45 | sec | 233 | sec | 465 | sec |
| S-Ethyl Tetrazole | 38.7 | 31 | μL | 45 | sec | 233 | min | 465 | sec |
| Acetic Anhydride | 655 | 124 | μL | 5 | sec | 5 | sec | 5 | sec |
| N-Methyl | 1245 | 124 | μL | 5 | sec | 5 | sec | 5 | sec |
| Imidazole | | | | | | | | | |
| TCA | 700 | 732 | μL | 10 | sec | 10 | sec | 10 | sec |
| Iodine | 20.6 | 244 | μL | 15 | sec | 15 | sec | 15 | sec |
| Beaucage | 7.7 | 232 | μL | 100 | sec | 300 | sec | 300 | sec |
| Acetonitrile | NA | 2.64 | mL | NA | | NA | | NA | |

| | | | | | |
|---|---|---|---|---|---|
| | Equivalents:DNA/ | Amount: DNA/2′-O- | | Wait Time* 2′-O- | Wait Time* |
| Reagent | 2′-O-methyl/Ribo | methyl/Ribo | Wait Time* DNA | methyl | Ribo |

C. 0.2 μmol Synthesis Cycle 96 well Instrument

| | | | | | | | | |
|---|---|---|---|---|---|---|---|---|
| Phosphoramidites | 22/33/66 | 40/60/120 μL | 60 | sec | 180 | sec | 360 | sec |
| S-Ethyl Tetrazole | 70/105/210 | 40/60/120 μL | 60 | sec | 180 | min | 360 | sec |
| Acetic Anhydride | 265/265/265 | 50/50/50 μL | 10 | sec | 10 | sec | 10 | sec |
| N-Methyl | 502/502/502 | 50/50/50 μL | 10 | sec | 10 | sec | 10 | sec |
| Imidazole | | | | | | | | |
| TCA | 238/475/475 | 250/500/500 μL | 15 | sec | 15 | sec | 15 | sec |
| Iodine | 6.8/6.8/6.8 | 80/80/80 μL | 30 | sec | 30 | sec | 30 | sec |
| Beaucage | 34/51/51 | 80/120/120 | 100 | sec | 200 | sec | 200 | sec |
| Acetonitrile | NA | 1150/1150/1150 μL | NA | | NA | | NA | |

*Wait time does not include contact time during delivery.

TABLE III
Human HER1-4 Receptor Hammerhead
Ribozyme and Substrate Sequence

| Substrate | Seq ID | Hammerhead Ribozyme | Seq ID |
|---|---|---|---|
| GUGAUGU C UGGAGCU | 8 | AGCUCCA CUGAUGAGGCCGUUAGGCCGAA ACAUCAC | 215 |
| GACUGCU U UGCCUGC | 24 | GCAGGCA CUGAUGAGGCCGUUAGGCCGAA AGCAGUC | 216 |
| UCAUGGU C AAAUGUU | 30 | AACAUUU CUGAUGAGGCCGUUAGGCCGAA ACCAUGA | 217 |
| CAAAUGU U GGAUGAU | 36 | AUCAUCC CUGAUGAGGCCGUUAGGCCGAA ACAUUUG | 218 |
| CACAGAU U UUGGGCU | 48 | AGCCCAA CUGAUGAGGCCGUUACGCCGAA AUCUGUG | 219 |
| ACAUGAU C AUGGUCA | 53 | UGACCAU CUGAUGAGGCCGUUAGGCCGAA AUCAUGU | 220 |
| GACCUCU C CUUCCUG | 62 | CAGGAAG CUGAUGAGGCCGUUAGGCCGAA AGAGGUC | 221 |
| UGGGAGU U GAUGACC | 70 | GGUCAUC CUGAUGAGGCCGUUAGGCCGAA ACUCCCA | 222 |
| GAUGUCU A CAUGAUC | 71 | GAUCAUG CUGAUGAGGCCGUUAGGCCGAA AGACAUC | 223 |
| GAUGUC U ACAUG | 81 | CAUGU CUGAUGAGGCCGUUAGGCCGAA GACAUC | 224 |
| GAUGU C UACAU | 91 | AUGUA CUGAUGAGGCCGUUAGGCCGAA ACAUC | 225 |
| UCCUU C CUGC | 118 | GCAG CUGAUGAGGCCGUUAGGCCGAA AAGGA | 226 |
| AUGUC U ACAU | 126 | AUGU CUGAUGAGGCCGUUAGGCCGAA GACAU | 227 |
| GUCU A CAUG | 133 | CAUG CUGAUGAGGCCGUUAGGCCGAA AGAC | 228 |
| AGAU U UUGG | 139 | CCAA CUGAUGAGGCCGUUAGGCCGAA AUCU | 229 |
| CCCU C CUCC | 152 | GGAG CUGAUGAGGCCGUUAGGCCGAA AGGG | 230 |
| CCUU C CUGC | 157 | GCAG CUGAUGAGGCCGUUAGGCCGAA AAGG | 231 |
| AGAU C ACAG | 160 | CUGU CUGAUGAGGCCGUUAGGCCGAA AUCU | 232 |
| GCUU C UUCA | 168 | UGAA CUGAUGAGGCCGUUAGGCCGAA AAGC | 233 |
| ACCU C UCCU | 175 | AGGA CUGAUGAGGCCGUUAGGCCGAA AGGU | 234 |
| CCCU C AGCC | 182 | GGCU CUGAUGAGGCCGUUAGGCCGAA AGGG | 235 |
| AUGU C UACA | 192 | UGUA CUGAUGAGGCCGUUAGGCCGAA ACAU | 236 |
| UGAU C AUGG | 198 | CCAU CUGAUGAGGCCGUUAGGCCGAA AUCA | 237 |
| GAUU C CAGU | 204 | ACUG CUGAUGAGGCCGUUAGGCCGAA AAUC | 238 |
| UCCU U CCUG | 210 | CAGG CUGAUGAGGCCGUUAGGCCGAA AGGA | 239 |

Underlined region can be any X sequence or linker, as described herein.

TABLE IV
Human HER1-4 Receptor Inozyme and Substrate Sequence

| Substrate | Seq ID | Inozyme | Seq ID |
|---|---|---|---|
| UGGAUGC U GAGGAGU | 1 | ACUCCUC CUGAUGAGGCCGUUAGGCCGAA ICAUCCA | 240 |
| CAUGGUC A AAUGUUG | 6 | CAACAUU CUGAUGAGGCCGUUAGGCCGAA IACCAUG | 241 |
| CACAGAC U GCUUUGC | 14 | GCAAAGC CUGAUGAGGCCGUUAGGCCGAA IUCUGUG | 242 |
| UGUCUAC A UGAUCAU | 18 | AUGAUCA CUGAUGAGGCCGUUAGGCCGAA IUAGACA | 243 |
| AGAUCAC A GGUUACC | 20 | GGUAACC CUGAUGAGGCCGUUAGGCCGAA IUGAUCU | 244 |
| GACAACC C UGACUAC | 23 | GUAGUCA CUGAUGAGGCCGUUAGGCCGAA IGUUGUC | 245 |
| CAAUGAC A GUGGAGC | 25 | GCUCCAC CUGAUGAGGCCGUUAGGCCGAA IUCAUUG | 246 |
| GCCAUCC A AACUGCA | 33 | UGCAGUU CUGAUGAGGCCGUUAGGCCGAA IGAUGGC | 247 |
| ACCCACC A GAGUGAU | 35 | AUCACUC CUGAUGAGGCCGUUAGGCCGAA IGUGGGU | 248 |
| ACCUCUC C UUCCUGC | 39 | GCAGGAA CUGAUGAGGCCGUUAGGCCGAA IAGAGGU | 249 |
| CAGUGAC U GCUGCCA | 42 | UGGCAGC CUGAUGAGGCCGUUAGGCCGAA IUCACUG | 250 |
| UGAUGUC U GGAGCUA | 43 | UAGCUCC CUGAUGAGGCCGUUAGGCCGAA IACAUCA | 251 |
| AGACUGC U UUGCCUG | 47 | CAGGCAA CUGAUGAGGCCGUUAGGCCGAA ICAGUCU | 252 |
| CAUGAUC A UGGUCAA | 55 | UUGACCA CUGAUGAGGCCGUUAGGCCGAA IAUCAUG | 253 |
| AGGACAC A GACUGCU | 68 | AGCAGUC CUGAUGAGGCCGUUAGGCCGAA IUGUCCU | 254 |
| UGCCAUC C AAACUGC | 72 | GCAGUUU CUGAUGAGGCCGUUAGGCCGAA IAUGGCA | 255 |
| AUGUC U ACAUG | 93 | CAUGU CUGAUGAGGCCGUUAGGCCGAA IACAU | 256 |
| GACCU C UCCU | 106 | AGGA CUGAUGAGGCCGUUAGGCCGAA IGGUC | 257 |
| UGUCU A CAUG | 112 | CAUG CUGAUGAGGCCGUUAGGCCGAA IGACA | 258 |
| UGACU G CUGC | 117 | GCAG CUGAUGAGGCCGUUAGGCCGAA IGUCA | 259 |
| CACCA G AGUG | 119 | CACU CUGAUGAGGCCGUUAGGCCGAA IGGUG | 260 |
| CUGC A CCCA | 136 | UGGG CUGAUGAGGCCGUUAGGCCGAA ICAG | 261 |
| UGUC U ACAU | 137 | AUGU CUGAUGAGGCCGUUAGGCCGAA IACA | 262 |
| CUGC U GCCA | 141 | UGGC CUGAUGAGGCCGUUAGGCCGAA ICAG | 263 |
| UUGC C AAGG | 148 | CCUU CUGAUGAGGCCGUUAGGCCGAA ICAA | 264 |
| CCCC A GCAG | 154 | CUGC CUGAUGAGGCCGUUAGGCCGAA IGGG | 265 |
| GACC U CUCC | 155 | GGAG CUGAUGAGGCCGUUAGGCCGAA IGUC | 266 |
| GACC C CCAG | 166 | CUGG CUGAUGAGGCCGUUAGGCCGAA IGUC | 267 |
| UGAC U GCUG | 188 | CAGC CUGAUGAGGCCGUUAGGCCGAA IUCA | 268 |
| UGCC C ACUG | 199 | CAGU CUGAUGAGGCCGUUAGGCCGAA IGCA | 269 |
| GUGC C ACCC | 200 | GGGU CUGAUGAGGCCGUUAGGCCGAA ICAC | 270 |
| CACC A GAGU | 206 | ACUC CUGAUGAGGCCGUUAGGCCGAA IGUG | 271 |

Underlined region can be any X sequence or linker, as described herein. I = Inosine

TABLE V
Human EGFR Receptor Zinzyme and Substrate Sequence

| Substrate | Seq ID | Zinzyme | Seq ID |
|---|---|---|---|
| CAGCAGG G CUUCUUC | 4 | GAAGAAG GCCGAAAGGCGAGUGAGGUCU CCUGCUG | 272 |
| GUCAAAU G UUGGAUG | 22 | CAUCCAA GCCGAAAGGCGAGUGAGGUCU AUUUGAC | 273 |
| UUUGGGA G UUGAUGA | 52 | UCAUCAA GCCGAAAGGCGAGUGAGGUCU UCCCAAA | 274 |
| GAGUGAU G UCUGGAG | 54 | CUCCAGA GCCGAAAGGCGAGUGAGGUCU AUCACUC | 275 |
| CUUCAGU G UUUUUUC | 58 | GAAAAAA GCCGAAAGGCGAGUGAGGUCU ACUGAAG | 276 |
| ACAGACU G CUUUGCC | 63 | GGCAAAG GCCGAAAGGCGAGUGAGGUCU AGUCUGU | 277 |
| CACCAGA G UGAUGUC | 73 | GACAUCA GCCGAAAGGCGAGUGAGGUCU UCUGGUG | 278 |
| ACCAGA G UGAUGU | 76 | ACAUCA GCCGAAAGGCGAGUGAGGUCU UCUGGU | 279 |
| CCAGAG U GAUGU | 83 | ACAUC GCCGAAAGGCGAGUGAGGUCU CUCUGG | 280 |
| UGACUG C UGCCA | 85 | UGGCA GCCGAAAGGCGAGUGAGGUCU CAGUCA | 281 |
| UGACU G CUGCC | 88 | GGCAG GCCGAAAGGCGAGUGAGGUCU AGUCA | 282 |
| CCAGA G UGAUG | 101 | CAUCA GCCGAAAGGCGAGUGAGGUCU UCUGG | 283 |
| CAGAG U GAUG | 104 | CAUC GCCGAAAGGCGAGUGAGGUCU CUCUG | 284 |
| GACUG C UGCC | 108 | GGCA GCCGAAAGGCGAGUGAGGUCU CAGUC | 285 |
| AACUG C ACCC | 116 | GGGU GCCGAAAGGCGAGUGAGGUCU CAGUU | 286 |
| AACU G CACC | 131 | GGUG GCCGAAAGGCGAGUGAGGUCU AGUU | 287 |
| CUGG G CUCC | 134 | GGAG GCCGAAAGGCGAGUGAGGUCU CCAG | 288 |
| UGCU G CCAU | 142 | AUGG GCCGAAAGGCGAGUGAGGUCU AGCA | 289 |
| GGCU C CUGG | 147 | CCAG GCCGAAAGGCGAGUGAGGUCU AGCC | 290 |
| GACU C CUGC | 149 | GCAG GCCGAAAGGCGAGUGAGGUCU AGUC | 291 |
| CAGU G UUUU | 151 | AAAA GCCGAAAGGCGAGUGAGGUCU ACUG | 292 |
| AGCU G CCCC | 158 | GGGG GCCGAAAGGCGAGUGAGGUCU AGCU | 293 |
| CCCU G CCCU | 162 | AGGG GCCGAAAGGCGAGUGAGGUCU AGGG | 294 |
| CUGG G CCAG | 167 | CUGG GCCGAAAGGCGAGUGAGGUCU CCAG | 295 |
| CUUU G UGGU | 177 | ACCA GCCGAAAGGCGAGUGAGGUCU AAAG | 296 |
| GCCU C UCCU | 183 | AGGA GCCGAAAGGCGAGUGAGGUCU AGGC | 297 |
| CAUG G UCAA | 194 | UUGA GCCGAAAGGCGAGUGAGGUCU CAUG | 298 |
| CAGA G UGAU | 197 | AUCA GCCGAAAGGCGAGUGAGGUCU UCUG | 299 |
| CGGA G CCCA | 212 | UGGG GCCGAAAGGCGAGUGAGGUCU UCCG | 300 |
| CAGA C CCCC | 213 | GGGG GCCGAAAGGCGAGUGAGGUCU UCUG | 301 |

TABLE VI
Human EGFR Receptor DNAzyme and Substrate Sequence

| Substrate | Seq ID | DNAzyme | Seq ID |
|---|---|---|---|
| CAGCAGG G CUUCUUC | 4 | GAAGAAG GGCTAGCTACAACGA CCTGCTG | 302 |
| GGAGUUG A UGACCUU | 5 | AAGGTCA GGCTAGCTACAACGA CAACTCC | 303 |
| AUGUUGG A UGAUUGA | 10 | TCAATCA GGCTAGCTACAACGA CCAACAT | 304 |
| CUACAUG A UCAUGGU | 11 | ACCATGA GGCTAGCTACAACGA CATGTAG | 305 |
| UGUCUAC A UGAUCAU | 18 | ATGATCA GGCTAGCTACAACGA GTAGACA | 306 |
| GACACAG A CUGCUUU | 21 | AAAGCAG GGCTAGCTACAACGA CTGTGTC | 307 |
| GUCAAAU G UUGGAUG | 22 | CATCCAA GGCTAGCTACAACGA ATTTGAC | 308 |
| AUCACAG A UUUUGGG | 34 | CCCAAAA GGCTAGCTACAACGA CTGTGAT | 309 |
| UGGUCAA A UGUUGGA | 49 | TCCAACA GGCTAGCTACAACGA TTGACCA | 310 |
| CAUCCAA A CUGCACC | 51 | GGTGCAG GGCTAGCTACAACGA TTGGATG | 311 |
| UUUGGGA G UUGAUGA | 52 | TCATCAA GGCTAGCTACAACGA TCCCAAA | 312 |
| GAGUGAU G UCUGGAG | 54 | CTCCAGA GGCTAGCTACAACGA ATCACTC | 313 |
| CAUGAUC A UGGUCAA | 55 | TTGACCA GGCTAGCTACAACGA GATCATG | 314 |
| CUUCAGU G UUUUUUC | 58 | GAAAAAA GGCTAGCTACAACGA ACTGAAG | 315 |
| CAGAGUG A UGUCUGG | 59 | CCAGACA GGCTAGCTACAACGA CACTCTG | 316 |
| ACAGACU G CUUUGCC | 63 | GGCAAAG GGCTAGCTACAACGA AGTCTGT | 317 |
| UUCAAUG A CAGUGGA | 65 | TCCACTG GGCTAGCTACAACGA CATTGAA | 318 |
| GAUGUCU A CAUGAUC | 71 | GATCATG GGCTAGCTACAACGA AGACATC | 319 |
| CACCAGA G UGAUGUC | 73 | GACATCA GGCTAGCTACAACGA TCTGGTG | 320 |
| UGUUGGA U GAUUGA | 75 | TCAATC GGCTAGCTACAACGA TCCAACA | 321 |
| ACCAGA G UGAUGU | 76 | ACATCA GGCTAGCTACAACGA TCTGGT | 322 |
| UGUUGG A UGAUUG | 77 | CAATCA GGCTAGCTACAACGA CCAACA | 323 |
| GUUGGA U GAUUG | 80 | CAATC GGCTAGCTACAACGA TCCAAC | 324 |
| CCAGAG U GAUGU | 83 | ACATC GGCTAGCTACAACGA CTCTGG | 325 |
| UGACUG C UGCCA | 85 | TGGCA GGCTAGCTACAACGA CAGTCA | 326 |
| UGACU G CUGCC | 88 | GGCAG GGCTAGCTACAACGA AGTCA | 327 |
| GUUGG A UGAUU | 97 | AATCA GGCTAGCTACAACGA CCAAC | 328 |
| CCAGA G UGAUG | 101 | CATCA GGCTAGCTACAACGA TCTGG | 329 |
| UUGGA U GAUU | 103 | AATC GGCTAGCTACAACGA TCCAA | 330 |
| CAGAG U GAUG | 104 | CATC GGCTAGCTACAACGA CTCTG | 331 |
| GACUG C UGCC | 108 | GGCA GGCTAGCTACAACGA CAGTC | 332 |
| AACUG C ACCC | 116 | GGGT GGCTAGCTACAACGA CAGTT | 333 |
| AACU G CACC | 131 | GGTG GGCTAGCTACAACGA AGTT | 334 |
| GUCU A CAUG | 133 | CATG GGCTAGCTACAACGA AGAC | 335 |
| CUGG G CUCC | 134 | GGAG GGCTAGCTACAACGA CCAG | 336 |
| CUGC A CCCA | 136 | TGGG GGCTAGCTACAACGA GCAG | 337 |
| AAUG A CAGU | 140 | ACTG GGCTAGCTACAACGA CATT | 338 |
| UGCU G CCAU | 142 | ATGG GGCTAGCTACAACGA AGCA | 339 |
| UUGG A UGAU | 144 | ATCA GGCTAGCTACAACGA CCAA | 340 |
| GAGA A UGUG | 145 | CACA GGCTAGCTACAACGA TCTC | 341 |
| GGCU G CUGG | 147 | CCAG GGCTAGCTACAACGA AGCC | 342 |
| GACU G CUGC | 149 | GCAG GGCTAGCTACAACGA AGTC | 343 |
| CAGU G UUUU | 151 | AAAA GGCTAGCTACAACGA ACTG | 344 |
| AGCU G CCCC | 158 | GGGG GGCTAGCTACAACGA AGCT | 345 |
| CCCU G CCCU | 162 | AGGG GGCTAGCTACAACGA AGGG | 346 |
| GAUG A CCUU | 164 | AAGG GGCTAGCTACAACGA CATC | 347 |
| CUGG G CCAG | 167 | CTGG GGCTAGCTACAACGA CCAG | 348 |
| AUUG A UGUC | 173 | GACA GGCTAGCTACAACGA CAAT | 349 |
| CUUU G UGGU | 177 | ACCA GGCTAGCTACAACGA AAAG | 350 |
| GCCU G UCCU | 183 | AGGA GGCTAGCTACAACGA AGGC | 351 |
| GUGG A UGGC | 189 | GCCA GGCTAGCTACAACGA CCAC | 352 |
| GAUG A UUGA | 190 | TCAA GGCTAGCTACAACGA CATC | 353 |
| CAUG G UCAA | 194 | TTGA GGCTAGCTACAACGA CATG | 354 |
| CAGA G UGAU | 197 | ATCA GGCTAGCTACAACGA TCTG | 355 |
| CGGA G CCCA | 212 | TGGG GGCTAGCTACAACGA TCCG | 356 |
| CAGA G CCCC | 213 | GGGG GGCTAGCTACAACGA TCTG | 357 |

TABLE VII
Human EGFR Receptor Amberzyme and Substrate Sequence

| Substrate | Seq ID | Amberzyme | Seq ID |
|---|---|---|---|
| CAGCAGG G CUUCUUC | 4 | GAAGAAG GGAGGAAACUCC CU UCAAGGACAUCGUCCGGG CCUGCUG | 358 |
| UCUACAU G AUCAUGG | 9 | CCAUGAU GGAGGAAACUCC CU UCAAGGACAUCGUCCGGG AUGUAGA | 359 |
| GGGAGUU G AUGACCU | 15 | AGGUCAU GGAGGAAACUCC CU UCAAGGACAUCGUCCGGG AACUCCC | 360 |
| AAUGUUG G AUGAUUG | 17 | CAAUCAU GGAGGAAACUCC CU UCAAGGACAUCGUCCGGG CAACAUU | 361 |
| GUCAAAU G UUGGAUG | 22 | CAUCCAA GAGGGAAACUCC CU UCAAGGACAUCGUCCGGG AUUUGAC | 362 |
| GGACACA G ACUGCUU | 26 | AAGCAGU GGAGGAAACUCC CU UCAAGGACAUCGUCCGGG UGUGUCC | 363 |
| GGAUGCU G AGGAGUA | 29 | UACUCCU GGAGGAAACUCC CU UCAAGGACAUCGUCCGGG AGCAUCC | 364 |
| CCAGAGU G AUGUCUG | 41 | CAGACAU GGAGGAAACUCC CU UCAAGGACAUCGUCCGGG ACUCUGG | 365 |
| AAAUGUU G GAUGAUU | 50 | AAUCAUC GGAGGAAACUCC CU UCAAGGACAUCGUCCGGG AACAUUU | 366 |
| UUUGGGA G UUGAUGA | 52 | UCAUCAA GGAGGAAACUCC CU UCAAGGACAUCGUCCGGG UCCCAAA | 367 |
| GAGUGAU G UCUGGAG | 54 | CUCCAGA GGAGGAAACUCC CU UCAAGGACAUCGUCCGGG AUCACUC | 368 |
| GAUCACA G GUUACCU | 56 | AGGUAAC GGAGGAAACUCC CU UCAAGGACAUCGUCCGGG UGUGAUC | 369 |
| CUUCAGU G UUUUUUC | 58 | GAAAAAA GGAGGAAACUCC CU UCAAGGACAUCGUCCGGG ACUGAAG | 370 |
| CAAGAUG G AAGUAGA | 60 | UCUACUU GGAGGAAACUCC CU UCAAGGACAUCGUCCGGG CAUCUUG | 371 |
| ACAGACU G CUUUGCC | 63 | GGCAAAG GGAGGAAACUCC CU UCAAGGACAUCGUCCGGG AGUCUGU | 372 |
| CCCACCA G AGUGAUG | 66 | CAUCACU GGAGGAAACUCC CU UCAAGGACAUCGUCCGGG UGGUGGG | 373 |
| GUUGGAU G AUUGAUG | 67 | CAUCAAU GGAGGAAACUCC CU UCAAGGACAUCGUCCGGG AUCCAAC | 374 |
| CACCAGA G UGAUGUC | 73 | GACAUCA GGAGGAAACUCC CU UCAAGGACAUCGUCCGGG UCUGGUG | 375 |
| ACCAGA G UGAUGU | 76 | ACAUCA GGAGGAAACUCC CU UCAAGGACAUCGUCCGGG UCUGGU | 376 |
| CCAGAG U GAUGU | 83 | ACAUC GGAGGAAACUCC CU UCAAGGACAUCGUCCGGG CUCUGG | 377 |
| UGACUG C UGCCA | 85 | UGGCA GGAGGAAACUCC CU UCAAGGACAUCGUCCGGG CAGUCA | 378 |
| CACCAG A GUGAU | 86 | AUCAC GGAGGAAACUCC CU UCAAGGACAUCGUCCGGG CUGGUG | 379 |
| UGUUGG A UGAUU | 87 | AAUCA GGAGGAAACUCC CU UCAAGGACAUCGUCCGGG CCAACA | 380 |
| UGACU G CUGCC | 88 | GGCAG GGAGGAAACUCC CU UCAAGGACAUCGUCCGGG AGUCA | 381 |
| UGGAU G AUUGA | 89 | UCAAU GGAGGAAACUCC CU UCAAGGACAUCGUCCGGG AUCCA | 382 |
| AAGUG G AUGGC | 94 | GCCAU GGAGGAAACUCC CU UCAAGGACAUCGUCCGGG CACUU | 383 |
| UGUUG G AUGAU | 96 | AUCAU GGAGGAAACUCC CU UCAAGGACAUCGUCCGGG CAACA | 384 |
| CACCA G AGUGA | 98 | UCACU GGAGGAAACUCC CU UCAAGGACAUCGUCCGGG UGGUG | 385 |
| CCAGA G UGAUG | 101 | CAUCA GGAGGAAACUCC CU UCAAGGACAUCGUCCGGG UCUGG | 386 |
| CAGAG U GAUG | 104 | CAUC GGAGGAAACUCC CU UCAAGGACAUCGUCCGGG CUCUG | 387 |
| GACUG C UGCC | 108 | GGCA GGAGGAAACUCC CU UCAAGGACAUCGUCCGGG CAGUC | 388 |
| GGAUG A UUGA | 109 | UCAA GGAGGAAACUCC CU UCAAGGACAUCGUCCGGG CAUCC | 389 |
| AAGUG G AUGG | 114 | CCAU GGAGGAAACUCC CU UCAAGGACAUCGUCCGGG CACUU | 390 |
| AGUGG A UGGC | 115 | GCCA GGAGGAAACUCC CU UCAAGGACAUCGUCCGGG CCACU | 391 |
| AACUG C ACCC | 116 | GGGU GGAGGAAACUCC CU UCAAGGACAUCGUCCGGG CAGUU | 392 |
| AAUGG A GACC | 120 | GGUC GGAGGAAACUCC CU UCAAGGACAUCGUCCGGG CCAUU | 393 |
| GUUGG A UGAU | 122 | AUCA GGACGAAACUCC CU UCAAGGACAUCGUCCGGG CCAAC | 394 |
| UGAUG A CCUU | 123 | AAGG GGAGGAAACUCC CU UCAAGGACAUCGUCCGGG CAUCA | 395 |
| UGUUG G AUGA | 128 | UCAU GGAGGAAACUCC CU UCAAGGACAUCGUCCGGG CAACA | 396 |
| ACCAG A GUGA | 129 | UCAC GGAGGAAACUCC CU UCAAGGACAUCGUCCGGG CUGGU | 397 |
| UCAUG G UCAA | 130 | UUGA GGAGGAAACUCC CU UCAAGGACAUCGUCCGGG CAUGA | 398 |
| AACU C CACC | 131 | GGUG GGAGGAAACUCC CU UCAAGGACAUCGUCCGGG AGUU | 399 |
| CUGG G CUCC | 134 | GGAG GGAGGAAACUCC CU UCAAGGACAUCGUCCGGG CCAG | 400 |
| GGAC G AUUG | 135 | CAAU GGAGGAAACUCC CU UCAAGGACAUCGUCCGGG AUCC | 401 |
| UGCU G CCAU | 142 | AUGG GGAGGAAACUCC CU UCAAGGACAUCGUCCGGG AGCA | 402 |
| CCGA G ACCC | 143 | GGGU GGAGGAAACUCC CU UCAAGGACAUCGUCCGGG UCGG | 403 |
| GGCU G CUGG | 147 | CCAG GGAGGAAACUCC CU UCAAGGACAUCGUCCGGG AGCC | 404 |
| GACU G CUGC | 149 | GCAG GGAGGAAACUCC CU UCAAGGACAUCGUCCGGG AGUC | 405 |
| AAUG G AGAC | 150 | GUCU GGAGGAAACUCC CU UCAAGGACAUCGUCCGGG CAUU | 406 |
| CAGU G UUUU | 151 | AAAA GGAGGAAACUCC CU UCAAGGACAUCGUCCGGG ACUG | 407 |
| AGCC G GAGC | 153 | GCUC GGAGGAAACUCC CU UCAAGGACAUCGUCCGGG GGCU | 408 |
| AACU G GUGU | 156 | ACAC GGACGAAACUCC CU UCAAGGACAUCGUCCGGG AGUU | 409 |
| AGCU G CCCC | 158 | GGGG GGAGGAAACUCC CU UCAAGGACAUCGUCCGGG AGCU | 410 |
| CAGU G GAGC | 159 | GCUC GGAGGAAACUCC CU UCAAGCACAUCGUCCGGG ACUG | 411 |
| UGAA G GAAA | 161 | UUUC GGACGAAACUCC CU UCAAGGACAUCGUCCGGG UUCA | 412 |
| CCCU G CCCU | 162 | AGGG GGAGGAAACUCC CU UCAACCACAUCGUCCGGG AGGG | 413 |
| UCAU G GUCA | 163 | UGAC GGAGGAAACUCC CU UCAAGGACAUCGUCCGGG AUGA | 414 |
| CUGG G CCAG | 167 | CUGG GGAGGAAACUCC CU UCAAGGACAUCGUCCGGC CCAG | 415 |
| UGUU G GAUG | 170 | CAUC GGAGGAAACUCC CU UCAAGGACAUCGUCCGGC AACA | 416 |
| GUUG G AUGA | 176 | UCAU GGAGGAAACUCC CU UCAACGACAUCGUCCGGG CAAC | 417 |
| CUUU G UGGU | 177 | ACCA GGAGCAAACUCC CU UCAAGGACAUCCUCCGGG AAAC | 418 |
| AACU G GAUG | 178 | CAUC GGAGGAAACUCC CU UCAAGGACAUCGUCCGGG ACUU | 419 |
| ACCA G AGUG | 180 | CACU GCAGGAAACUCC CU UCAAGGACAUCGUCCCGG UGGU | 420 |
| GCCU G UCCU | 183 | AGGA GCAGGAAACUCC CU UCAAGGACAUCGUCCCGG AGGC | 421 |
| UGCU G GGGU | 184 | ACCC GCAGGAAACUCC CU UCAAGGACAUCGUCCCGG AGCA | 422 |
| AGUG G AUGG | 187 | CCAU GCAGGAAACUCC CU UCAAGGACAUCGUCCCGG CACU | 423 |
| CAUG G UCAA | 194 | UUGA GCAGGAAACUCC CU UCAAGGACAUCGUCCCGG CAUG | 424 |
| AGAU G GAGG | 195 | CCUC GCAGGAAACUCC CU UCAAGGACAUCGUCCCGG AUCU | 425 |
| CAGA G UGAU | 197 | AUCA GCAGGAAACUCC CU UCAAGGACAUCGUCCCGG UCUG | 426 |
| CACA G ACUG | 207 | CAGU GCAGGAAACUCC CU UCAAGGACAUCGUCCCGG UGUG | 427 |
| GAGU G AUGU | 208 | ACAU GCAGGAAACUCC CU UCAAGGACAUCGUCCCGG ACUC | 428 |
| UGAU G ACCU | 211 | AGGU GCAGGAAACUCC CU UCAAGGACAUCGUCCCGG AUCA | 429 |
| CGGA G CCCA | 212 | UGGG GCAGGAAACUCC CU UCAAGGACAUCGUCCCGG UCCG | 430 |
| CAGA G CCCC | 213 | GGGG GCAGGAAACUCC CU UCAAGGACAUCGUCCCGG UCUG | 431 |
| UGAG G AGUA | 214 | UACU GCAGGAAACUCC CU UCAAGGACAUCGUCCCGG CUCA | 432 |

What we claim is:

1. An enzymatic nucleic acid molecule which down regulates expression of an epidermal growth factor receptor (EGFR) gene, wherein said EGFR gene comprises HER1, HER2, HER3, or HER4 and any combination thereof.

2. An enzymatic nucleic acid molecule comprising a sequence having SEQ ID NOs: 215-432.

3. An enzymatic nucleic acid molecule comprising at least one binding arm wherein one or more of said binding arms comprises a sequence complementary to a sequence having SEQ ID NOs: 1-214.

4. An antisense nucleic acid molecule comprising a sequence complementary to a sequence having SEQ ID NOs: 1-214.

5. The enzymatic nucleic acid of any of claims 1-3, wherein said enzymatic nucleic acid molecule is adapted to treat cancer.

6. The antisense nucleic acid of claim 4, wherein said antisense nucleic acid molecule is adapted to treat cancer.

7. The enzymatic nucleic acid molecule of any of claims 1-3, wherein said enzymatic nucleic acid molecule has an endonuclease activity to cleave RNA encoded by an EGFR gene.

8. The enzymatic nucleic acid molecule of claim 7, wherein said EGFR gene comprises HER1, HER2, HER3, or HER4 and any combination thereof.

9. The enzymatic nucleic acid molecule of claim 1, wherein said enzymatic nucleic acid molecule is in a hammerhead configuration.

10. The enzymatic nucleic acid molecule of claim 1, wherein said enzymatic nucleic acid molecule is in an Inozyme configuration.

11. The enzymatic nucleic acid molecule of claim 1, wherein said enzymatic nucleic acid molecule is in a Zinzyme configuration.

12. The enzymatic nucleic acid molecule of claim 1, wherein said enzymatic nucleic acid molecule is in a DNAzyme configuration.

13. The enzymatic nucleic acid molecule of claim 1, wherein said enzymatic nucleic acid molecule is in a G-cleaver configuration.

14. The enzymatic nucleic acid molecule of claim 1, wherein said enzymatic nucleic acid molecule is in an Amberzyme configuration.

15. The enzymatic nucleic acid molecule of claim 9, wherein said hammerhead configuration comprises a sequence complementary to a sequence having SEQ ID NOs: 8, 24, 30, 36, 48, 53, 62, 70, 71, 81, 91, 118, 126, 133, 139, 152, 157, 160, 168, 175, 182, 192, 198, 201, and 210.

16. The enzymatic nucleic acid molecule of claim 9, wherein said hammerhead configuration comprises a sequence having SEQ ID NOs: 215-239.

17. The enzymatic nucleic acid molecule of claim 10, wherein said Inozyme configuration comprises a sequence complementary to a sequence having SEQ ID NOs: 1, 6, 14, 18, 20, 23, 25, 33, 35, 39, 42, 43, 47, 55, 68, 72, 93, 106, 112, 117, 119, 136, 137, 141, 148, 154, 155, 166, 188, 199, 200, and 206.

18. The enzymatic nucleic acid molecule of claim 10, wherein said Inozyme configuration comprises a sequence having SEQ ID NOs: 240-271.

19. The enzymatic nucleic acid molecule of claim 11, wherein said Zinzyme configuration comprises a sequence complementary to a sequence having SEQ ID NOs: 4, 22, 52, 54, 58, 63, 73, 76, 83, 85, 88, 101, 104, 108, 116, 131, 134, 142, 147, 149, 151, 158, 162, 167, 177, 183, 194, 197, 212, and 213.

20. The enzymatic nucleic acid molecule of claim 11, wherein said Zinzyme configuration comprises a sequence having SEQ ID NOs: 272-301.

21. The enzymatic nucleic acid molecule of claim 12, wherein said DNAzyme configuration comprises a sequence complementary to a sequence having SEQ ID NOs: 4, 5, 10, 11, 18, 21, 22, 34, 49, 51, 52, 54, 55, 58, 59, 63, 65, 71, 73, 75, 76, 77, 80, 83, 85, 88, 97, 101, 103, 104, 108, 116, 131, 133, 134, 136, 140, 142, 144, 145, 147, 149, 151, 158, 162, 164, 167, 173, 177, 183, 189, 190, 194, 197, 212, and 213.

22. The enzymatic nucleic acid molecule of claim 12, wherein said DNAzyme configuration comprises a sequence having SEQ ID NOs: 302-357.

23. The enzymatic nucleic acid molecule of claim 13, wherein said Amberzyme configuration comprises a sequence complementary to a sequence having SEQ ID NOs: 4, 9, 15, 17, 22, 26, 29, 41, 50, 52, 54, 56, 58, 60, 63, 66, 67, 73, 76, 83, 85, 86, 87, 88, 89, 94, 96, 98, 101, 104, 108, 109, 114, 115, 116, 120, 122, 123, 128, 129, 130, 131, 134, 135, 142, 143, 147, 149, 150, 151, 153, 156, 158, 159, 161, 162, 163, 167, 170, 176, 177, 178, 180, 1783, 184, 187, 194, 195, 197, 207, 208, 211-214.

24. The enzymatic nucleic acid molecule of claim 13, wherein said Amberzyme configuration comprises a sequence having of SEQ ID NOs: 358-432.

25. The enzymatic nucleic acid molecule of any of claims 1-3, wherein said enzymatic nucleic acid molecule comprises between 8 and 100 bases complementary to the RNA of EGFR gene.

26. The enzymatic nucleic acid molecule of any of claims 1-3, wherein said enzymatic nucleic acid molecule comprises between 14 and 24 bases complementary to the RNA of EGFR gene.

27. The enzymatic nucleic acid molecule of any of claims 1-3, wherein said enzymatic nucleic acid molecule is chemically synthesized.

28. The antisense nucleic acid molecule of claim 4, wherein said antisense nucleic acid molecule is chemically synthesized.

29. The enzymatic nucleic acid molecule of any of claims 1-3, wherein said enzymatic nucleic acid molecule comprises at least one 2′-sugar modification.

30. The antisense nucleic acid molecule of claim 4, wherein said antisense nucleic acid molecule comprises at least one 2′-sugar modification.

31. The enzymatic nucleic acid molecule of any of claims 1-3, wherein said enzymatic nucleic acid molecule comprises at least one nucleic acid base modification.

32. The antisense nucleic acid molecule of claim 4, wherein said antisense nucleic acid molecule comprises at least one nucleic acid base modification.

33. The enzymatic nucleic acid molecule of any of claims 1-3, wherein said enzymatic nucleic acid molecule comprises at least one phosphate backbone modification.

34. The antisense nucleic acid molecule of claim 4, wherein said antisense nucleic acid molecule comprises at least one phosphate backbone modification.

35. A mammalian cell including the enzymatic nucleic acid molecule of any of claims 1-3.

36. The mammalian cell of claim 35, wherein said mammalian cell is a human cell.

37. A method of reducing EGFR activity in a cell, comprising contacting said cell with the enzymatic nucleic acid molecule of any of claims 1-3, under conditions suitable for said reduction.

38. A method of reducing EGFR activity in a cell, comprising contacting said cell with the antisense nucleic acid molecule of claim 4 under conditions suitable for said reduction.

39. A method of treatment of a patient having a condition associated with the level of EGFR, comprising contacting cells of said patient with the enzymatic nucleic acid molecule of any of claims 1-3, under conditions suitable for said treatment.

40. A method of treatment of a patient having a condition associated with the level of EGFR, comprising contacting cells of said patient with the antisense nucleic acid molecule of claim 4, under conditions suitable for said treatment.

41. The method of claim 37 further comprising the use of one or more drug therapies under conditions suitable for said treatment.

42. The method of claim 38 further comprising the use of one or more drug therapies under conditions suitable for said treatment.

43. The method of claim 39 further comprising the use of one or more drug therapies under conditions suitable for said treatment.

44. The method of claim 40 further comprising the use of one or more drug therapies under conditions suitable for said treatment.

45. A method of cleaving RNA of EGFR gene comprising contacting an enzymatic nucleic acid molecule of any of claims 1-3 with said RNA of EGFR gene under conditions suitable for the cleavage.

46. The method of claim 45, wherein said cleavage is carried out in the presence of a divalent cation.

47. The method of claim 46, wherein said divalent cation is Mg²⁺.

48. The enzymatic nucleic acid molecule of any of claims 1-3, wherein said enzymatic nucleic acid comprises a cap structure, wherein the cap structure is at the 5′-end, or 3′-end, or both the 5′-end and the 3′-end.

49. The antisense nucleic acid molecule of claim 4, wherein said antisense nucleic acid comprises a cap structure, wherein the cap structure is at the 5′-end, or 3′-end, or both the 5′-end and the 3′-end.

50. The enzymatic nucleic acid molecule of claim 48, wherein the cap structure at the 5′-end, 3′-end, or both the 5′-end and the 3′-end comprises a 3′,3′-linked or 5′,5′-linked deoxyabasic ribose derivative.

51. The antisense nucleic acid molecule of claim 49, wherein the cap structure at the 5′-end, 3′-end, or both the 5′-end and the 3′-end comprises a 3′,3′-linked or 5′,5′-linked deoxyabasic ribose derivative.

52. The method of claim 37, wherein said enzymatic nucleic acid molecule is in a Zinzyme configuration.

53. An expression vector comprising a nucleic acid sequence encoding at least one enzymatic nucleic acid molecule of claim 1 or claim 3 in a manner which allows expression of the nucleic acid molecule.

54. A mammalian cell including an expression vector of claim 53.

55. The mammalian cell of claim 54, wherein said mammalian cell is a human cell.

56. The expression vector of claim 53, wherein said enzymatic nucleic acid molecule is in a hammerhead configuration.

57. The expression vector of claim 53, wherein said expression vector further comprises a sequence for an antisense nucleic acid molecule complementary to the RNA of EGFR gene.

58. The expression vector of claim 53, wherein said expression vector comprises a nucleic acid sequence encoding two or more of said enzymatic nucleic acid molecules, which may be the same or different.

59. The expression vector of claim 58, wherein said expression vector further comprises a sequence encoding an antisense nucleic acid molecule complementary to the RNA of EGFR gene.

60. A method for treatment of cancer comprising the step of administering to a patient the enzymatic nucleic acid molecule of any of claims 1-3 under conditions suitable for said treatment.

61. The method of claim 60, wherein said cancer is breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, lymphoma, glioma, or multidrug resistant cancer.

62. A method for treatment of cancer comprising administering to a patient the antisense nucleic acid molecule of claim 4 under conditions suitable for said treatment.

63. The method of claim 62, wherein said cancer is breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, lymphoma, glioma, or multidrug resistant cancer.

64. The method of claim 60, wherein said enzymatic nucleic acid molecule is in a Zinzyme configuration.

65. The method of claim 60, wherein said method further comprises administering to said patient one or more other treatment therapies.

66. The method of claim 62, wherein said method further comprises administering to said patient one or more other treatment therapies.

67. The enzymatic nucleic acid molecule of claim 1 or claim 3, wherein said enzymatic nucleic acid molecule comprises at least five ribose residues, at least ten 2′-O-methyl modifications, and a 3′-end modification.

68. The enzymatic nucleic acid molecule of claim 67, wherein said enzymatic nucleic acid molecule further comprises phosphorothioate linkages on at least three of the 5′ terminal nucleotides.

69. The nucleic acid molecule of claim 67, wherein said 3′-end modification is a 3′-3′ inverted abasic moiety.

70. The method of claim 41 wherein said other drug therapies are monoclonal antibodies, EGFR-specific tyrosine kinase inhibitors, or chemotherapy.

71. The method of claim 70, wherein said monoclonal antibodies comprise mAB IMC C225 and mAB ABX-EGF.

72. The method of claim 70, wherein said EGFR-specific tyrosine kinase inhibitors comprise OSI-774 and ZD1839.

73. The method of claim 70, wherein said chemotherapy is paclitaxel, docetaxel, cisplatin, methotrexate, cyclophosphamide, doxorubin, fluorouracil carboplatin, edatrexate, gemcitabine, or vinorelbine.

74. The method of claim 42 wherein said other drug therapies are monoclonal antibodies, EGFR-specific tyrosine kinase inhibitors, or chemotherapy.

75. The method of claim 74, wherein said monoclonal antibodies comprise mAB IMC C225 and mAB ABX-EGF.

76. The method of claim 74, wherein said EGFR-specific tyrosine kinase inhibitors comprise OSI-774 and ZD1839.

77. The method of claim 74, wherein said chemotherapy is paclitaxel, docetaxel, cisplatin, methotrexate, cyclophosphamide, doxorubin, fluorouracil carboplatin, edatrexate, gemcitabine, or vinorelbine.

78. The method of claim 43 wherein said other drug therapies are monoclonal antibodies, EGFR-specific tyrosine kinase inhibitors, or chemotherapy.

79. The method of claim 78, wherein said monoclonal antibodies comprise mAB IMC C225 and mAB ABX-EGF.

80. The method of claim 78, wherein said EGFR-specific tyrosine kinase inhibitors comprise OSI-774 and ZD1839.

81. The method of claim 78, wherein said chemotherapy is paclitaxel, docetaxel, cisplatin, methotrexate, cyclophosphamide, doxorubin, fluorouracil carboplatin, edatrexate, gemcitabine, or vinorelbine.

82. The method of claim 44 wherein said other drug therapies are monoclonal antibodies, EGFR-specific tyrosine kinase inhibitors, or chemotherapy.

83. The method of claim 82, wherein said monoclonal antibodies comprise mAB IMC C225 and mAB ABX-EGF.

84. The method of claim 82, wherein said EGFR-specific tyrosine kinase inhibitors comprise OSI-774 and ZD1839.

85. The method of claim 82, wherein said chemotherapy is paclitaxel, docetaxel, cisplatin, methotrexate, cyclophosphamide, doxorubin, fluorouracil carboplatin, edatrexate, gemcitabine, or vinorelbine.

86. The method of claim 65, wherein said other treatment therapies are monoclonal antibodies, EGFR-specific tyrosine kinase inhibitors (TKIs), chemotherapy, or radiation therapy.

87. The method of claim 86, wherein said monoclonal antibodies comprise mAB IMC C225 and mAB ABX-EGF.

88. The method of claim 86, wherein said EGFR-specific tyrosine kinase inhibitors comprise OSI-774 and ZD1839.

89. The method of claim 86, wherein said chemotherapy is paclitaxel, docetaxel, cisplatin, methotrexate, cyclophosphamide, doxorubin, fluorouracil carboplatin, edatrexate, gemcitabine, or vinorelbine.

90. The method of claim 66, wherein said other treatment therapies are monoclonal antibodies, EGFR-specific tyrosine kinase inhibitors (TKIs), chemotherapy, or radiation therapy.

91. The method of claim 90, wherein said monoclonal antibodies comprise mAB IMC C225 and mAB ABX-EGF.

92. The method of claim 90, wherein said EGFR-specific tyrosine kinase inhibitors comprise OSI-774 and ZD1839.

93. The method of claim 90, wherein said chemotherapy is paclitaxel, docetaxel, cisplatin, methotrexate, cyclophosphamide, doxorubin, fluorouracil carboplatin, edatrexate, gemcitabine, or vinorelbine.

94. A pharmaceutical composition comprising an enzymatic nucleic acid molecule of any of claims 1-3.

95. A pharmaceutical composition comprising an antisense nucleic acid molecule of claim 4.

96. A method of administering to a mammal the enzymatic nucleic acid molecule of claim 1, comprising contacting said mammal with the compound under conditions suitable for said administration.

97. The method of claim 96, wherein said mammal is a human.

98. The method of claim 96 wherein said administration is in the presence of a delivery reagent.

99. The method of claim 98, wherein said delivery reagent is a lipid.

100. The method of claim 99, wherein said lipid is a cationic lipid.

101. The method of claim 99, wherein said lipid is a phospholipid.

102. The method of claim 98, wherein said delivery reagent is a liposome.
